(19) United States
(12) Patent Application Publication
(10) Pub. No.: US 2026/0041620 A1
(43) Pub. Date: Feb. 12, 2026

(54) NOVEL AMINO ACID BASED SALTS, COMPOSITIONS AND METHODS FOR TREATING SKIN AND IMPROVING SKIN REJUVENATION AND LONGEVITY

(71) Applicant: Sytheon Ltd, Parsippany, NJ (US)

(72) Inventor: Ratan K Chaudhuri, Lincoln Park, NJ (US)

(73) Assignee: Sytheon Ltd, Parsippany, NJ (US)

(21) Appl. No.: 19/360,143

(22) Filed: Oct. 16, 2025

Related U.S. Application Data
(62) Division of application No. 18/775,629, filed on Jul. 17, 2024.
(60) Provisional application No. 63/527,483, filed on Jul. 18, 2023.
(60) Provisional application No. 63/621,598, filed on Jan. 17, 2024.

(51) Int. Cl. A61K 8/44; A61K 8/34; A61K 8/35; A61K 8/49; A61K 8/9789; A61Q 19/00; A61Q 19/08

(52) U.S. Cl. CPC A61K 8/44; A61K 8/347; A61K 8/35; A61K 8/4973; A61K 8/9789; A61Q 19/007; A61Q 19/08

(57) ABSTRACT

Select substituted lysine and branched amino acid salts, compositions containing the same and the use of the foregoing in treating the adverse physiological effects of various diseases, especially skin diseases; in mitigating the effects of aging and environmental factors on skin; and in improving and rejuvenating skin.

### RELATED APPLICATIONS

The present application is a divisional application of U.S. patent application Ser. No. 18/775,629, filed Jul. 17, 2024, entitled “Novel Amino Acid Based Salts, Compositions and Methods for Treating Skin and Rejuvenating Skin and Extending Longevity” which claims the benefit of prior United States Provisional Patent Application Nos. 63/527,483, filed Jul. 18, 2023, entitled “Novel Amino Acid Based Histone Deacetylase Modulators for Treating Skin” and 631621,598, filed Jan. 17, 2024, entitled “Compositions and Methods for Regulating Malanogenesis,” the contents of both of which are hereby incorporated herein by reference in their entirety.

### TECHNICAL FIELD

The present disclosure describes select lysine and branched amino acid salts, their analogs, tautomeric forms, stereoisomers, polymorphs, and hydrates as well as pharmaceutically and cosmetically acceptable divalent metal salts, compositions, metabolites, and prodrugs thereof. These compounds have been found to inhibit or activate, depending upon the compound, a number of enzymes, including, histone deacetylase (HDAC) enzymes; maintain stemness; elongate telomeres; and boost regenerates collagens as well as to be useful as therapeutic or ameliorating agents for preventing and/or reversing signs of skin aging; for improving wound healing; for improving barrier function and repair and skin hydration; for controlling pigmentation, inflammation and immunosenescence; and for mitigating, reversing and/or preventing the manifestation of adverse effects on the skin due to various skin diseases and physiological and environmental factors.

### BACKGROUND

Histones are among the most abundant proteins in the body. They are highly basic proteins abundant in lysine and arginine residues that are found in eukaryotic cell nuclei. They act as spools that help compact DNA so our enormous genomes will fit in the tight space of the nucleus. Each spool is made of eight histone proteins and encircled by just 147 of the human genome's more than 3 billion base pairs, meaning each cell needs more than 163 million histones to hold base pairs. Amazingly, although the DNA is very tightly folded, it is compacted in a way that allows it to easily become available to the many enzymes in the cell that replicate it, repair it, and use its genes to produce proteins [B Albetds et. al., Molecular Biology of the Cell. 4th edition, New York: Garland Science, 2002].

Among all known histone modifications, acetylation has the highest potential to induce chromatin unfolding, as it neutralizes the electro-static interaction between the histone and the negatively charged DNA, making it more accessible to the transcriptional apparatus [H Zentner, Regulation of nucleosome dynamics by histone modification, Nat Struc Mol Biol, 20:259-266, 2013]. In normal cells, there is a fine balance between histone acetylation and deacetylation [E Roperto, The role of histone deacetylases in human cancers, Mol Oncol, 1:19-25, 2007], This balance is primarily controlled by HDACs and histone acetyl transferases (HATs) [Gong et al, Acetylation readers protein: linking acetylation signaling to genome maintenance and cancer, PLoS Genet, 12, e1006273, 2016; Shen et al., Histone acetylation enzymes coordinate metabolism and gene expression, Trends Plan Sci, 20:614-621 2015]. HATs catalyze the transfer of the acetyl moiety from acetyl coenzyme A to the ε-amino groups of histone lysine residues, thereby neutralizing the positive charge of histone tails. This results in a more open chromatin state and greater access of DNA to transcription factors, HDACs, on the contrary, catalyze the removal of acetyl groups from the lysine residues of histone tails, resulting in a more condensed, transcriptionally repressive chromatin conformation [Peng et al., Histone deacetylase activity assay. Methods Mol Biol, 1268:95-108, 2015]. Overall, histone acetylation and deacetylation play a crucial role in modifying chromatin structure and thus regulating gene expression, cell proliferation, migration, and apoptosis, as well as immune functions and angiogenesis [Chen et al., Histone deacetylases and mechanisms of regulation of gene expression, Crit Rev Oncog, 20:35-47, 2015; Zhou et al., Role of histone deacetylases in vascular cell homeostasis and artioclerosis, Cardivasc Res, 90:413-420, 2011]. Overall, the removal of acetyl groups has profound effects on gene expression, Global gene expression profiling experiments estimate that the transcription of 10% of genes are regulated by histone deacetylases.

We shall keep in mind the following facts while we discuss Histone Deacetylases (HDACs) and histone deacetylase inhibitors (HDACis). (i) HDACs deacetylate not only histones, but also non-histone proteins [B N Singh et al., Nonhistone protein acetylation as cancer therapy targets. Expert Rev. Anticancer Ther. 10, 935-954, 2010; M A Glozak et. al., Acetylation and deacetylation of non-histone proteins. Gene. 363, 15-23, 2005]. (ii) HDACs can also catalyze other deacylation reactions, such as demalonylation, desuccinylation, or decrotonylation or debutyralation [W Li and Z Sun, Mechanism of Action for HDAC Inhibitors—Insights from Omics Approaches Int J Mol Sci. 20(7), 1616, 2019; https://doi.org/10.3390/ijms20071616: references cited therein]. (iii) HDACs have enzyme-independent functions [W Li and Z Sun, Mechanism of Action for HDAC Inhibitors-insights from Omics Approaches Int J Mol Sci. 20(7), 1616, 2019; https.//doi.org/10.3390/ijms20071616 references cited therein]. Human histone deacetylases (HDACs) are categorized into four classes that are based on sequence homology [E Seto and M Yoshida, Erasers of histone acetylation: The histone deacetylase enzymes. Cold Spring Harb Perspect Biol. 6, a018713, 2014]. Class I includes HDAC 1, 2, 3, and 8; class IIa includes HDAC 4, 5, 7, and 9; class IIb includes HDAC 6, and 10; class IV includes HDAC 11; and class Ill includes SIRT1-7 [E Seto and M Yoshida, Erasers of histone acetylation: The histone deacetylase enzymes. Cold Spring Har Perspect Biol 6, a018713, 2014]. Class I, II, and IV are canonical zinc dependent HDACs and are simply referred to as HDACs. Class III are NAD-dependent sirtuins that are more distantly related to HDAC.

Transcriptional regulation is a major event in cell differentiation, proliferation, and apoptosis. Transcriptional activation of a set of genes determines cellular function and is tightly regulated by a variety of factors. One of the regulatory mechanisms involved in this process is an alteration in the tertiary structure of DNA, which affects transcription factors to their target DNA regiments. Nucleosomal integrity is regulated by the acetylation status of the core histone, with the result being permissiveness to transcription. The regulations of transcription factor are thought to involve changes in the structure of chromatin. Changing the affinity of histone proteins for coiled DNA in the nucleosome alters the structure of chromatin. Hypoacetylated histones are believed to have greater affinity to the DNA and form a tightly bound DNA-histone complex and render the DNA inaccessible to transcriptional regulation. The acetylating status of the histone is governed by the balanced activities of the histone acetyl transferase (HAT) and histone deacetylase (HDAC).

It is known that histone deacetylase inhibitors (HDACi) block HDAC enzyme activity by binding to the zinc ion in the catalytic site, which blocks substrate access to the site [J Melesina et al, Strategies to Design Selective Histone Deacetylase inhibitors, Chem Med Chem, 2021; https://doi.org/10.1002/cmdc.202000934; A Vannini et al., Substrate binding to histone deacetylases as shown by the crystal structure of the HDAC8-substrate complex. EMBQ Rep. 8, 879-884, 2007; B E Lauffer et al. Histone deacetylase (HDAC) inhibitor kinetic rate constants correlate with cellular histone acetylation but not transcription and cell viability. J. Biol Chem. 288, 26926-26943, 2013]. HDACi usually consists of a zinc binding group, surface binding group, and a linker that connects the above two components and spans the hydrophobic catalytic site channel [A Vannini et al, Substrate binding to histone deacetylases as shown by the crystal structure of the HDAC8-substrate complex. EMBO Rep. 8, 879-884, 2007; B E Lauffer et al. Histone deacetylase (HDAC) inhibitor kinetic rate constants correlate with cellular histone acetylation but not transcription and cell viability. J. Biol Chem. 288, 26926-26943, 2013]. HDACis can be categorized into groups that are based on their chemical nature [W Li and Z Sun, Mechanism of Action for HDAC Inhibitors—Insights from Omics Approaches Int J Mol Sci. 20(7), 1616, 2019; https://doi.org/10.3390/ijms20071616: references cited therein], Hydroxymates, such as suberoylanilide hydroxamic acid (SAHA) (vorinostat), TSA (trichostatin A), LBH589 (panobinostat), and PXD101 (belinostat), are pan-HDACis that inhibit all HDACs. Short-chain fatty acids, such as VPA (valproic acid) and butyrate, inhibit class I and Ila HDACs. Benzamides, such as MS275 (entinostat), and depsipeptides, such as FK228 (romidepsin), inhibit some of the class I HDACs. Cyclic tetrapeptide, such as TPX (trapoxin), target some class I, Ila and IV HDAGs. ACY-1215 (ricolinostat) is a selective inhibitor for HDAC6 [W Li and Z Sun, Mechanism of Action for HDAC Inhibitors—Insights from Omics Approaches Int J Mol Sci. 20(7), 1616, 2019; https://doi.org/10.3390/ijms20071616; references cited therein].

Many acetylation markers on histones decrease with age, including bulk histone 4 acetylation levels and histone 3 acetylation at lysine residues 18, 27, and 56, which is thought to facilitate the aging process (Feser J, Tyler J, Chromatin structure as a mediator of aging, FEBS Lett, 2011, 585:2041-2048). Therefore, the effects of an HDAC inhibitor, which prevents HDACs from removing acetyl groups further, have the potential to directly reverse or prevent these age-related changes. The role of epigenetic alterations in aging can be interconnected with other hallmarks, broadening the reach of HDAC inhibitors positively benefit aging at the molecular level.

The cellular homeostasis of proteins involves (i) their biogenesis by ribosomes, (ii) their folding by chaperones, and (iii) their degradation by proteasomes and autophagy. Inhibition of HDACs may benefit aging at all three steps of proteostasis. Once proteins are synthesized by ribosomes, multiple quality control mechanisms ensure their stability and functionality, including protein chaperones such as the heat shock proteins (HSPs). The heat shock response plays a beneficial role in lifespan regulation (Hsu A L, Murphy C T, Kenyon C, Regulation of aging and age-related disease by DAF-16 and heat-shock factor, Science, 2003, 300: 1142-1145). The last step in proteostasis involves the decomposition of proteins, performed either by proteasomal degradation or autophagy, both of which play key roles in aging and can be regulated by HDACs. This suggests HDAC inhibitors may contribute to lifespan extension also by protein quality assurance pathways. These findings suggest that HDAC inhibition provides benefits at all steps required for proteostasis, and directly acts to ameliorate this hallmark of aging, Intervening mitochondrial biology can mitigate skin aging.

With age, several mitochondrial regulatory factors diminish, leading to mitochondria with a decreased capacity for energy generation, as well as increased accumulation of damage and reduced mitochondrial turnover. There is strong evidence that HDAC inhibitors can prevent or reverse some of this deterioration, Butyrate has been demonstrated in several studies to elevate mitochondrial biogenesis, leading to increases in oxygen consumption (Galmozzi A, et al., Inhibition of class I histone deacetylases unveils a mitochondrial signature and enhances oxidative metabolism in skeletal muscle and adipose tissue, Diabetes, 2013, 62: 732-742), (Walsh M E, Van Remmen H (2016) Emerging roles for histone deacetylases in age-related muscle atrophy, Nutr Healthy Aging, 2016 4: 17-30).

Inducing senescence, a quiescent, non-dividing cell state, in cancerous cells is a desired outcome for cancer therapy, and several studies have shown HDAC inhibitors to cause various cancer cells to senescence (Vargas J E et al, Inhibition of HDAC increases the senescence induced by natural polyphenols in glioma cells, 2014, Biochem Cell Biol, 2014, 92:297-304), In the context of aging however, senescent cell reduction, rather than promotion, is desired. Importantly, the ability of HDAC inhibitors to cause cell senescence may be cancer specific; sodium butyrate was shown to potentiate senescence in human and rat glioma cell lines but not in normal astrocytes (Vargas J E et al., Inhibition of HDAC increases the senescence induced by natural polyphenols in glioma cells, 2014, Biochem Cell Biol, 2014, 92:297-304). However, the potential benefit of HDAC inhibitors may provide to ameliorate the hallmark of cellular senescence which is an unexplored field.

The reach of HDACs is broad. Epidermal keratinocytes participate in immune defense through their capacity to recognize danger, trigger inflammation, and resist infection. However, normal skin immune function must tolerate contact with an abundant community of commensal microbes without unwanted inflammation that would promote disease [Alferink et al, Control of neonatal tolerance to tissue antigens by peripheral T cell trafficking. Science 282, 1338-1341, 1998; Zhang et al., Unique aspects of the perinatal immune system, Nat Rev Immunol, 17, 495-507, 2017]. For example, failure of immune tolerance mechanisms results in classic allergic responses to specific antigens and common human inflammatory skin disorders, such as psoriasis, atopic dermatitis and acne, reflect dysfunction of tolerance to innate immune stimuli. However, despite the importance of innate immune tolerance to overall immune homeostasis, and its potential relevance to multiple human diseases, the understanding and study of these mechanisms are still in their infancy.

A common mechanism for environmental control of cell function is through epigenetic control of gene expression. The microbial environmental conditions dictate the production of molecules that influence epigenetic events and cause keratinocytes to break innate immune tolerance. Cutibacterium acnes (C. acnes) is one such commensal skin bacterium and produces short-chain fatty acids (SCFAs), an important class of molecules involved in epigenetic control that can increase acetylation of histones through inhibition of histone deacetylases (HDACs). SCFAs are generated from bacteria under anaerobic conditions [Arpaia. et al., Metabolites produced by commensal bacteria promote peripheral regulatory T-cell generation, Nature, 504, 451-455, 2013] and can accumulate in hair follicles due to fermentation by C. acnes [Shu et al., Fermentation of Propionibacterium acnes, a commensal bacterium in the human skin microbiome, as skin probiotics against methicillin-resistant Staphylococcus aureus, PloS one 6, e55380, 2013]. Although SCFAs, such as butyrate, have been known to inhibit inflammatory responses by bone-marrow derived immunocytes [Arpaia, et. al., Metabolites produced by commensal bacteria promote peripheral regulatory T-cell generation, Nature, 504, 451-455, 2013; Furusawa Y et al, Commensal microbe-derived butyrate induces the differentiation of colonic regulatory T cells, Nature, 504, 446-450, 2013], an important clue to understand innate immune tolerance of the skin came with the observation that SCFAs increase inflammatory responses by keratinocytes to TLR ligands. Inhibition of HDAC8 and HDAC9 in keratinocytes by SCFAs produced by C. acnes amplified inflammatory responses in keratinocytes [Sanford et al., Inhibition of HDAC-8 and HDAC-9 by microbial short-chain fatty acids breaks immune tolerance of the eoidermis to TLR ligands, Science Immunology 1, eaah4609, 2016]. This response was opposite to the action of HDAC inhibition on production of inflammatory cytokines.

In addition to the inhibition of inflammatory responses, certain SCFAs, namely butyrate, has emerged to play a pivotal role in influencing the predominance of definite cutaneous microbiome profiles, which subsequently evoke skin immune defense mechanisms, by protecting against infection and ultraviolet radiation, and providing adequate nourishment to the cells of the skin [Salem I, et al., The Gut Microbiome as a Major Regulator of the Gut-Skin Axis. Front, Micribiol, 2018, 10:1459]. Indeed, it has been shown that butyric acid alone or Staphylococcus epidermidis with glycerol topical application remarkably ameliorated the UVB-induced inflammation on mice skin [Keshari S Balasubramaniam et al., Butyric Acid from Probiotic Staphylococcus epidermidis in the Skin Microbiome Down-Regulates the Ultraviolet-Induced Pro-inflammatory IL-6 Cytokine via Short-Chain Fatty Acid Receptor, Int J Mol Sci, 2019:11:4477]. Butyric acid noticeably decreased the ulceration of the skin and the epidermal thickness from UVB exposure and exerted a significant reduction in IL-6 and IL-8 level. These butyrate immunomodulatory effects are mediated by the binding with the GPR43, which controls the pro-inflammatory cytokines production elicited by skin injury [Keshari S Balasubramaniam et. al., Butyric Acid from Probiotic Staphylococcus epidermidis in the Skin Microbiome Down-Regulates the Ultraviolet-Induced Pro-Inflammatory IL-6 Cytokine via Short-Chain Fatty Acid Receptor, Int J Mol Sci, 2019; 11:4477]. Topical butyrate administration may become a useful therapeutic application with a “curative” potential on inflammatory skin diseases. Therefore, butyrate can be effective not only for overt skin diseases, but as an ingredient in cosmetic products, to prevent skin alterations. Unfortunately, the main limitation factor for the use of butyrate in dermatology is its unfavorable sensorial (very offensive odor) and physicochemical properties. Butyric acid esters are also very unstable and releases butyric acid even at room temperature.

Moreover, although Na butyrate has been reported to have HDAC inhibitory activity (IC₅₀ values are 0.3, 0.4 and 0.3 mM for HDAC1, 2 and 7, respectively), it shows no inhibition of HDAC6 and 10 (httpsl/www.abcam.com/sodium-butvrate-histone-deacetylase-inhibitor-ab120948.html). Furthermore, its short systemic half-life has limited its use as a systemic therapy [Miller A et al, Clinical pharmacology of sodium butyrate in patients with acute leukemia, Eur J Cancer Clin Onco, 1987; 23 (9) 1283-1287].

Following on the foregoing discussion of epigenetic control, several independent lines of evidence support the conclusion that MAP2K3 is directly affected by HDAC8 and HDAC9, thus providing epigenetic control of keratinocyte immune function. There are few reported studies focused on MAP2K3-related skin diseases [Li. et. al., The p38-MAPK/SAPK pathway is required for human keratinocyte migration on dermal collagen, J Invest Dermatol, 117, 1601-1611, 2001]. Previously published transcriptional data from human skin diseases showed expression of MAP2K3 increases in major inflammatory skin diseases such as atopic dermatitis, psoriasis, and acne vulgaris. This association is consistent with certain findings that regulation of MAP2K3 function may be a key checkpoint for maintaining homeostasis. Furthermore, additional studies have shown that inhibition of the MAP2K3 downstream target, p38MAPK, impairs inflammatory responses in IMO-induced psoriasis inflammation [Sakurai et. al., Cutaneous p38 mitogen-activated protein kinase activation triggers psoriatic dermatitis, J Allergy Clin Immunol, 144, 1036-1049, 2019], C. acnes-induced skin inflammation [Weber et at, Nivolumab versus chemotherapy in patients with advanced melanoma who progressed after anti-CTLA-4 treatment (CheckMate 037): a randomised, controlled, open-label, phase 3 trial Lancet Oncol 16, 375-384, 2015] and UV radiation-induced skin inflammation [Hildesheim et al., p38 Mitogen-activated protein kinase inhibitor protects the epidermis against the acute damaging effects of ultraviolet irradiation by blocking apoptosis and inflammatory responses, J Invest Dermatol, 122, 497-502, 2004].

Although structurally diverse classes of small-molecule HDACis have been identified, as noted above, many of the known HDACis feature strong Zn-chelating warheads that could lead to unintended off-target effects, particularly with respect to other metalloenzymes. Recently, a novel series of HDAC inhibitors belonging to cyclic peptides lacking a zinc-binding moiety has been developed by DC Beshore et al., [D C Beshore et al., Redefining the Histone Deacetylase Inhibitor Pharmacophore: High Potency with No Zinc Cofactor Interaction, ACS Med. Chem. Lett. 2021, 12, 4, 540-547], potentially mitigating the concern with such off-target interaction.

Though certainly significant, HDACs are not alone in contributing to skin aging and health issues. Chronic stress accelerates the visible signs of skin aging, including development of fine lines and deep wrinkles, uneven skin tone and laxity, with early indicators of a compromised skin barrier including the symptoms of uncomfortable skin such as visible redness, sensitivity, dryness and blemishes, One of the defenses our bodies possess is a natural ability to fight stress through the endocannabinoid system (ECS) which, when activated by stress, helps restore homeostasis and induce a calming effect. (T Biró et al., The endocannabinoid system of the skin in health and disease: novel perspectives and therapeutic opportunities, Trends Pharmacol Sci. 2009 August; 30(8): 411-420, 2009; S K Baswan et al., Therapeutic Potential of Cannabidiol (CBD) for Skin Health and Disorders, Clinical, Cosmetic and Investigational Dermatology, 13:927-942, 2020).

The endocannabinoid system (ECS) has multiple regulatory functions both in health and disease. Recent studies have intriguingly suggested the existence of a functional EQS in the skin and implicates it in various biological processes (e.g. proliferation, growth, differentiation, apoptosis and cytokine, mediator or hormone production of various cell types of the skin and appendages, such as the hair follicle and sebaceous gland), It seems that the main physiological function of the cutaneous ECS is to constitutively control the well-balanced proliferation, differentiation and survival, as well as immune competence and/or tolerance, of skin cells. The disruption of this delicate balance may result in multiple pathological conditions and diseases of the skin (e.g. acne, seborrhea, allergic dermatitis, itch and pain, psoriasis, hair growth disorders, systemic sclerosis and cancer).

Endocannabinoids are molecules made by our body as needed, Experts have identified Arachidonoyl ethanolamide (AEA, also known as Anandamide, the bliss molecule) and 2-arachidonoylglycerol (2-AG) are the key endocannabinoids. Enzymes are responsible for breaking down endocannabinoids once they have carried out their function. The degradation of AEA and 2-AG primarily is regulated by two enzymes, fatty acid amide hydrolase (FAAH) and monoacylglycerol lipase (MAGL), respectively. Certain transport proteins have also been found to play a key role in the endocannabinoid system. Fatty acid binding proteins, such as FABP-3 and FABP-5 appear to enhance cellular uptake of AEA or 2-AG, which promotes the hydrolysis of AEA or 2-AG into arachidonic acid thereby increasing inflammation. FABP-3 and 5 have additionally been shown to bind to arachidonic acid and mobilize it to cellular nuclei resulting in the production of pro-inflammatory gene expression.

The branched-chain amino acids (BCAAs), leucine, isoleucine, and valine, share a structurally similar side chain. Together with lysine, the BCAAs are essential amino acids (EAAs), meaning that mammals, including humans, must meet their metabolic needs via a sufficient dietary intake. The branched-chain amino acids (BCAAs) leucine, isoleucine, and valine, comprise ˜17% of human skeletal muscle. BCAAs fulfill 3 distinct roles. First, they provide building blocks for protein synthesis and promote protein synthesis. Second, when catabolized via the TCA (described above), they act as a fuel source. Finally, they stimulate cell signaling via activation of mTOR, which uncouples insulin signaling and regulates protein translation, thereby balancing cell growth and autophagy [Z Zhang et al. Branched-Chain Amino Acids as Critical Switches in Health and Disease, Z Zhang, Hypertension, 2018; 72:1012-1022]

Surprisingly, we have discovered a group of divalent salts, especially Zn²⁺ salts, of select amino acids, namely lysine and the branched amino acids leucine, isoleucine, and valine and their derivatives, which have HDAC modification/regulation capabilities, particularly HDAC inhibition properties.

In following, we have discovered salts and compositions containing the same that maintain stemness, elongate telomers, boost regeneration of collagens and are useful as therapeutic or ameliorating agents for preventing and/or reversing signs of skin aging; for improving wound healing; for improving barrier function and repair and skin hydration; for controlling pigmentation, inflammation and immunosenescence; and for mitigating, reversing and/or preventing the manifestation of adverse effects on the skin due to various skin diseases and physiological and environmental factors.

### SUMMARY

According to the present teaching there are provided compounds according to Structure I:

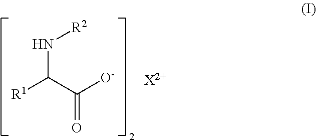

wherein:
X is Zn, Ca, Mg or Mn, preferably Zn;
each R¹ is independently selected from the group consisting of —(CH₂)₄NHR³, CH₂CH(CH₃)₂, —CH(CH₃)CH₂CH₃, and —CH(CH₃)₂;
each R² is independently selected from the group consisting of —H, —COCH₃, —COCH₂CH₃, —COCH₂CH₂CH₃, —COCH(CH₃)₂; —COCH₂CH₂COOCH₃; —COCH(COOCH₃)₂ and —COCH(CH₃)COOCH₃;
each R³ is independently selected from the group consisting of —H, —COCH₃, —COCH₂CH₃, —COCH₂CH₂CH₃, —COCH(CH₃)₂; —COCH₂CH₂COOCH₃; —COCH(COOCH₃)₂ and —COCH(CH₃)COOCH₃.

Preferably each R¹ is the same, each R² is the same and each R³ is the same. Also, it is preferred that in each compound, R¹ and R² are the same: though, as noted below, mixtures of said compounds are also allowed wherein the R¹ and R² of one compound are not the same as in another compound in the mixture.

According to a second aspect of the present teaching there are provided compositions consisting essentially of one or more compounds of Formula I, wherein the compounds are in the form of a salt (particularly the divalent salt), metabolite, analog, polymorph, tautomer, clathrate, stereoisomer, hydrate, solvate, or a prodrug thereof or a mixture of any two or more of the foregoing forms.

According to a third aspect of the present teaching there are provided compositions of matter comprising one or more compounds according to Formula I or one or more of its alternative forms as noted in the previous paragraph, particularly compositions of matter that are appropriate for administering to a human for the treatment of signs of skin aging, for improving wound healing, for improving barrier function and repair and skin hydration, for controlling pigmentation, inflammation and immunosenescence and for mitigating skin diseases.

According to a fourth aspect of the present teaching there are provided methods for treating signs of skin aging, for improving wound healing, for improving barrier function and repair and skin hydration, for controlling pigmentation, inflammation and immunosenescence and for mitigating skin diseases.

Such compositions typically include additional ingredients and/or carriers, particularly dermatologically acceptable and pharmaceutically acceptable carriers, alone or in combination with other suitable ingredients.

### DETAILED DESCRIPTION

For convenience, before further description of the present disclosure, certain terms employed in the specification, and examples are collected here. These definitions should be read in the light of the remainder of the disclosure and understood as would be understood by a person of skill in the art. While these terms are well recognized and known to those having skill in the art, for convenience and completeness, a general synopsis of the particular terms and their meanings are set forth below.

The articles “a”, “an” and “the” are used to refer to one or more than one (i.e, to at least one) of the grammatical object of the article.

The terms “amide forming functionality” and “amide forming functionalities” refers to those carbonyl-containing moieties which, when bonded to a nitrogen atom, form an amide or amide functional group.

The terms “comprise” and “comprising” are used in the inclusive, open sense, meaning that additional elements may be included. Throughout this specification, unless the context requires otherwise the word “comprise”, and variations, such as “comprises” and “comprising”, will be understood to imply the inclusion of a stated element or step or group of elements or steps but not the exclusion of any other element or step or group of elements or steps.

The term “including” is used to mean “including but not limited to” “including” and “including but not limited to” are used interchangeably.

Though the compounds and compositions disclosed herein are characterized as being in accordance with Structure I, presented as the salt, it is to be appreciated that the scope of the present teaching encompasses other forms of those structures/compounds. In following, the compounds described herein may contain one or more chiral centers and/or, therefore, may exist as stereoisomers, such as enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form, and mixtures thereof. Accordingly, the chemical structures depicted herein encompass all possible tautomeric forms of the illustrated or identified compounds. It is also understood that some isomeric form such as diastereomers, enantiomers and geometrical isomers can be separated by physical and/or chemical methods by those skilled in the art. Pharmaceutically acceptable solvates may be hydrates or comprising of other solvents of crystallization such as alcohols, ether, and the like. The term “solvates”, as used herein, refers to a crystal lattice which contains solvent. The term “hydrate” refers to a more specific form of solvate, wherein the solvent is water.

The term “polymorphs” refers to crystal forms of the same molecule, and different polymorphs may have different physical properties such as, for example, melting temperatures, heats of fusion, solubilities, dissolution rates and/or vibrational spectra due to the specific arrangement or conformation of the molecules in the crystal lattice.

The term “prodrugs” refers to the precursor of the compound of Structure 1, which on administration undergoes chemical conversion by metabolic processes before becoming active pharmacological substances. In general, such prodrugs will be functional derivatives of a compound of the invention, which are readily convertible in vivo into a compound of the invention as described below with the formation of Lysine derivatives for example (Scheme 1) and corresponding free acids, for example acetic acid, propionic acid, butyric acid and/or succinic acid monomethyl ether and their corresponding Zn²⁺ salts,

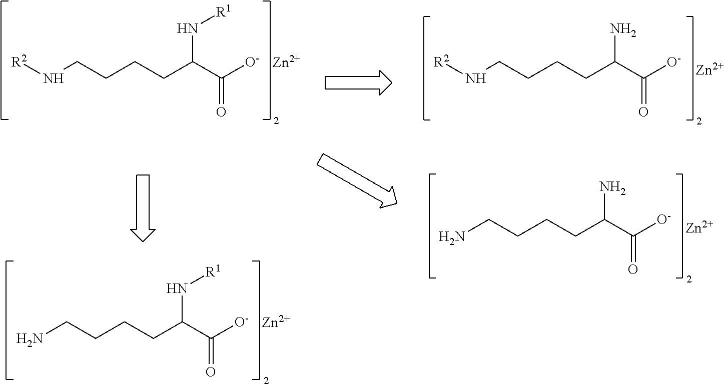

### Scheme 1: In-Vivo or In-Vitro Conversion of Products of Present Inventive Compositions

The terms “histone deacetylase” and “HDAC” are intended to refer to any one of a family of enzymes that remove acetyl groups from the ε-amino groups of lysine residues at the N-terminus of a histone or tubulin. Unless otherwise indicated by context, the term “histone” is meant to refer to any histone protein, including H1, H2A, H2B, H3, H4 and H5, from any species. Human HDAC proteins or gene products include but are not limited to, HDAC-1, HDAC-2, HDAC-3, HDAC-4, HDAC-5, HDAC-6, HDAC-7, HDAC-8, HDAC-9, HDAC-10 and HDAC-11.

The term “histone deacetylase inhibitor” or “inhibitor of histone deacetylase” is used to identify a compound, which can interact with a histone deacetylase and inhibit its activity, more particularly its enzymatic activity. Inhibiting histone deacetylase enzymatic activity means reducing the ability of a histone deacetylase to remove an acetyl group from a histone or tubulin. Preferably, such inhibition is specific, i.e., the histone deacetylase inhibitor reduces the ability of histone deacetylase to remove an acetyl group from a histone or tubulin at a concentration that is lower than the concentration of the inhibitor that is required to produce some other, unrelated biological effect.

A “dermatologically acceptable carrier” refers to a material that acts as a diluent, dispersant, vehicle or carrier for the stated actives and is recognized in the industry as acceptable or suitable for use, preferably long-term use, in skin contact and, most preferably, without undue toxicity, incompatibility, irritability, allergic response and the like. Typically, and to the extent appropriate or applicable, dermatologically acceptable carriers include those carriers that have been approved or are otherwise approvable by a regulatory agency of a government or governmental body or that are listed in the U.S. Pharmacopoeia or other generally recognized pharmacopoeia for use on humans. In the case of topical compositions, each composition will include any of the known topical excipients and agents necessary for achieving the particular form of the final composition, the selection of the carrier depended, in part upon the form of the topical composition, e.g., lotion, cream, gel, foam, emulsion, dispersion, spray, liposome, coacervate, etc. As used herein the term carrier also refers to base compositions used in formulating cosmetic, skin care, skin therapy and topical pharmaceutical products as well as such products themselves. Typically, the carrier will comprise from about 30 to more than 99 wt. %, preferably from 40 to 99 wt. %, of the composition.

Suitable dermatologically acceptable carriers are well known and include, e.g., mineral oils, silicone oils, emulsifying agents, water, alcohol, water and alcohol combinations, solvent, solvent systems, and combinations thereof. Additional suitable carriers and carrier compositions are described at length in, for example, Gonzalez et. al. —U.S. Pat. No. 7,186,404; Aust et. al.—U.S. Pat. No. 7,175,834; Roseaver et. al.—U.S. Pat. No. 7,172,754; Simoulidis et. al.—U.S. Pat. No. 7,175,835; Mongiat et. al.—U.S. Pat. No. 7,101,536; Maniscalco—U.S. Pat. No. 7,078,022; Forestier et. al, U.S. Pat. Nos. 5,175,340, 5,567,418, 5,538,716, and 5,951,968; Deflandre et. al.—U.S. Pat. No. 5,670,140; Chaudhuri—U.S. Pat. Nos. 7,150,876, 6,831,191, 6,602,515, 7,166,273, 6,936,735, 6,831,191, and 6,699,463; Chaudhuri et. al.—U.S. Pat. Nos. 6,165,450 and 7,150,876; Bonda et. al. U.S. Pat. No. 6,962,692; Rodan et. al.—U.S. Pat. No. 9,144,434, Wang et. al. U.S. Pat. No. 5,830,441 and Auspitz et. al.—US 2007/0110685 A.

The term “Pharmaceutically acceptable” means that the subject of this descriptor has been approved or is otherwise approvable by a regulatory agency of a government or governmental or is listed in the U.S. Pharmacopoeia or other generally recognized pharmacopoeia for use on or in humans.

A “pharmaceutically acceptable carrier” refers to a pharmaceutically acceptable diluent, a pharmaceutically acceptable adjuvant, a pharmaceutically acceptable excipient, a pharmaceutically acceptable vehicle or carrier, or a combination of any of the foregoing with which a pharmacological active agent, including the compounds provided by the present disclosure, can be administered to a patient, which does not destroy or have a marked adverse effect on the pharmacological activity of the therein contained pharmacological active agent or metabolite thereof and which is preferably non-toxic or of acceptable toxicity when administered in doses sufficient to provide a therapeutically effective amount of the pharmacological active agent or metabolite thereof. As would be appreciated by those skilled in the art, many, if not most of the aforementioned dermatologically acceptable carries are also suitable for use as the pharmaceutically acceptable carrier. Typically, the carrier will comprise from about 30 to more than 99 wt. %, preferably from 40 to 99 wt. %, of the composition.

A “pharmaceutical composition” refers to a composition comprising a pharmaceutically acceptable carrier and a pharmacological active agent or metabolite, especially, in the case of pharmaceutical compositions described in and claimed by the present application.

“Treating” or “treatment” of any disease or malady refers to reversing, alleviating, arresting, inhibiting, interfering and/or ameliorating the appearance and/or proliferation of a disease or at least one of the clinical symptoms of a disease, inhibiting the progress of a disease or at least one of the clinical symptoms of the disease as well as delaying the onset of a disease or at least one or more symptoms thereof in a patient who is predisposed to a disease, especially as evidenced by genetic testing, even though that patient does not yet experience or display symptoms of the disease. In following, treating or treatment also refers to inhibiting a disease, either physically, (e.g., stabilization of a discernible symptom), physiologically, (e.g., stabilization of a physical parameter), or both, and to inhibiting at least one physical parameter that may or may not be discernible to the patient.

“Improve” or “improvement” is used to convey the fact that the pharmacological active agent has manifested or effected changes, most notably beneficial changes, in either the characteristics and/or the physical attributes of the tissue to which it is being provided, applied, administered or targeted, including. e.g., the dermal-epidermal junction, necrosis of skin cancer cells, etc. These terms are also used to indicate that the symptoms or physical characteristics associated with the diseased state are diminished, reduced, or eliminated.

“Inhibiting” generally refers to delaying the onset of the symptoms, delaying, or stopping the progression of the disease and/or its manifestation symptoms, alleviating the symptoms, or eliminating the disease, condition, or disorder.

“Optional” or “optionally” means that the subsequently described subject, event or circumstance is not required or a necessary consequence, and that the description includes instances where the event occurs and instances where it does not and/or when the subject is present and when it is not present.

“Therapeutically effective amount” refers to the amount of a compound or composition that, when administered to a patient for treating a disease, or at least one of the clinical symptoms of a disease, is sufficient to affect such treatment of the disease or symptom thereof, including improvement of the afflicted organ or physiological process adversely affected by the disease or condition. The therapeutically effective amount varies depending, for example, on the compound or composition, the disease and/or symptoms of the disease, the severity of the disease and/or symptoms of the disease, the age, weight, and/or health of the patient to be treated, the mode of administration, the presence of synergistic agents, the judgment of the prescribing physician and the like. An appropriate amount of any given compound or composition can be ascertained by those skilled in the art and/or is capable of determination by routine experimentation.

“Therapeutically effective dose” refers to a dose that provides a therapeutically effective amount of the given agent to provide effective treatment of a disease in a patient. A therapeutically effective dose varies from compound/composition to compound/composition and/or from patient to patient and depends upon factors such as the condition of the patient and the route of delivery as well as those described in the preceding definition of therapeutically effective amount. A therapeutically effective dose can be determined in accordance with routine pharmacological procedures known to those skilled in the art, A therapeutic effective dose also contemplates the use of an initial or charging dose followed by sequent doses, daily, weekly or whatever, wherein the quantity of the active agent in the initial dose is greater than in the subsequent doses.

According to the teaching of the present application there are provided compounds according to Structure I as follows:

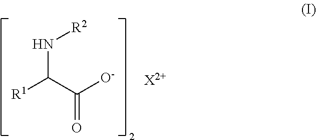

wherein:
X is Zn, Ca, Mg or Mn, preferably Zn;
each R¹ is independently selected from the group consisting of —(CH₂)₄NHR³, —CH₂CH(CH₃)₂, —CH(CH₃)CH₂CH₃, and —CH(CH₃)₂;
each R² is independently selected from the group consisting of —H, —COCH₁, —COCH₂CH₃, —COCH₂CH₂CH₃, —COCH(CH₃)₂; —COCH₂CH₂COOCH₃; —COCH(COCH₃)₂ and —COCH(CH₃)COOCH₃; preferably, —H, —COCH₃, —COCH₂CH₃, —OCH₂H₂CH₃, —COCH(CH₃);
each R³ is independently selected from the group consisting of —H, —COCH₃, —COCH₂CH₃, —COCH₂CH₂CH₃, —COCH(CH₃)₂; —COCH₂CH₂COCH₃; —COCH(COOCH₃)₂ and —COCH(CH₃)COOCH; preferably, —COCH₃, —COCH₂CH₃, —COCCHH₂CH₃, —CCH(CH₃)₂; most preferably, —COCH₂CH₂CH₃, —COCH(CH₃)₂.

Preferably each R¹ is the same, each R² is the same and each R³ is the same. Also, it is preferred that in each compound, R² and R³ are the same, more preferably that at least one of R² and R³ in each compound is not —H, i.e., at least one the two R²s or at least one of the two R³s is not —H, most preferably that none of R² and R³ are —H. The present teaching also encompasses mixtures of these compounds wherein the R¹s, R²s and/or R³s of one compound are not the same as in another compound in the mixture.

A preferred group of compounds according to the present teaching are those according to Structure II

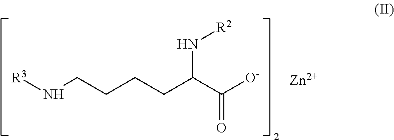

wherein R² and R³ are as defined above. Preferably, though not necessarily, at least one of the R² and R³ moieties is not —H; more preferably neither of the R² and R³ moieties is —H. Additionally, R² and R³ can be identical or non-identical: the latter meaning at least one of the R² and R³ moieties is —H and the other(s) an amide forming functionality or two different amide forming functionalities. Exemplary compounds according to Structure II include:
Compound 1: Zinc di-(diacetyl lysinate): R²═R³═—COCH₃.
Compound 2: Zinc di-(acetyl lysinate): one of R² and R³, preferably R³, is —COCH₃, and the other —H;
Compound 3: Zinc di-(dipropionyl lysinate): R²═R³═—COCH₂CH₃.
Compound 4: Zinc di-(propionyl lysinate): one of R² and R³, preferably R³, is —COCH₂CH, and the other —H;
Compound 5: Zinc di-(di-n-butyryl lysinate): R²═R³═—COCH₂CH₂CH₃.
Compound 6: Zinc di-(n-butyryl lysinate): one of R² and R³, preferably R³, is —COCH₂CH₂CH₃, and the other —H;
Compound 7: Zinc di-(di-isobutyryl lysinate): R²═R³═COCH(CH₃)₂.
Compound 8: Zinc di-(isobutyryl lysinate): one of R² and R³, preferably R³, is —COCH(CH₃)₂, and the other —H;

Compound 9: Zinc Di-(dimethyl succinyl lysinate): R²═R³═COCH₂CH₂COOCH₃.

Compound 10: Zinc Di-(dimethyl succinyl lysinate): one of R² and R³, preferably R, is —COCH₂CH₂COOCH₃, and the other —H;

Compound 11: Zinc Di-lysinate: R²═R³═H.

A preferred and especially beneficial group of compounds according to Structures I and II are those wherein at least one of the R² and, if present, the R³ moieties are butyrate moieties, n-butyryl or isobutyryl, most preferably n-butyryl: most preferably, when R³ is present, both R² and R³ are butyrate moieties, again, most preferably n-butyryl. As noted in the Background above, butyrate can be an effective active ingredient for topical health and beauty products and pharmaceuticals for certain skin conditions, but its use is curtailed, if not prevented by, its odor. Surprisingly, the odor problem does not manifest with the butyrated compounds of Structure 1. Specifically, these compounds are odorless and potent HDAC inhibitors. Even with the slow release of butyric acid from the breakdown/metabolism/dissociation of these compounds, no offensive odor is manifested, Additionally, these compounds are much more effective HDAC inhibitors than Na butyrate.

While the compounds of the present teaching are preferably in the form of the di-valent salts of Structures I and II, depending upon the methodology of their production, the mole ratio of the reactants and the like, these compounds and compositions consisting essentially of these compounds are created in a number of forms and mixtures of such forms including the mono-valent salts; the divalent salts; as metabolites, analogs, polymorphs, tautomers, clathrates, stereoisomers, hydrates, solvates, and prodrugs or a combination of any two of more of the foregoing, Additionally, and/or alternatively, it may be desirable for a given application and/or performance to convert the compounds or salts or the product of their production into one of these alternate forms. For convenience in discussing compositions comprising these compounds and compositions consisting essentially of said compounds as well as applications for the forgoing and methods of use, reference hereinafter to these compounds or the compounds of the present teaching is to be understood as embracing both these compounds as well as the various forms thereof and the compositions consisting essentially of the foregoing. In addition, the scope of the present teaching also encompasses the monovalent salts of Structures I and II as well as those structures wherein the amino acid function may not be fully neutralized with the Zn, Ca, Mg or Mn ion.

The compounds of the present teaching, namely those according to Structure I above, most especially those of Structure II above, may serve as the primary component, especially the primary active component, of various compositions and products or it may be incorporated into a number of different compositions and products as a supplemental/additional active and/or a synergistic active for the intended use of said compositions and products. Given the impact of these compounds on HDAC and other physiological processes and the benefits associated therewith, these compounds are especially desirable as active pharmaceutical ingredients, therapeutic agents, cosmetic and skin care actives and the like.

In following, the present teaching also pertains to pharmaceutical and non-pharmaceutical topical compositions comprising at least one of the compounds of the present teaching, preferably those of Structures I and II wherein neither R² nor R³, if present, is —H, most preferably those compounds of Structure I wherein one or, preferably both, R² and R³ are n-butyryl or isobutyryl, most especially n-butyryl, in combination with a dermatologically acceptable carrier and/or a pharmaceutically acceptable carrier. The compounds of the present teaching are present in an effective amount for achieving the desired/intended outcome, a therapeutically effective amount in the case of pharmaceutical compositions. Exemplary non-pharmaceutical compositions include, for example, dedicated compositions for addressing the skin condition, improvement or other purpose for which it is to be employed as well as formulated cosmetic, health and beauty aid products, personal care products and/or OTC health products. Similarly, these compounds may be used in the production and formulation of topical pharmaceuticals, especially as APIs, for use in the treatment of various maladies and diseases and/or the symptoms thereof, affecting the skin. In such cases, these compounds are typically formulated into a pharmaceutically acceptable carrier in an effective amount. Generally, an effective amount in both instances is from about 0.01 to about 10 percent, preferably from about 0.1 to about 10 percent, more typically from about 0.1 to about 5 percent, by weight based on the total weight of the final composition.

The topical formulations or products produced with these compounds may take many forms depending upon the use, method of use or application and the like. For example, the topical formulations containing these materials may be in the form of a cream, an ointment, a gel, a paste, a powder, a spray, a suspension, a dispersion, a solution, a salve, a lotion, a patch, a liposome formation, a microcapsule, a nanocapsule, liquid, serum, foam, liquid foundation, balm antiperspirant, emulsions, dispersions, coacervates, pads, swabs, wipes, patches, sponges, and, as applicable, combinations thereof. Depending upon the application and the delivery mechanism or form, the formulations or products can further contain a penetration enhancing agent.

Additionally, the compositions and products containing the compounds of the present teaching, particularly consumer and, as applicable, pharmaceutical products, formulated therewith, may include one or more additional ingredients such as antioxidants, sunscreens, skin lightening actives, exfoliants, anti-acne actives, vitamins, anti-inflammatory agents, self-tanning agents, moisturizers, emollients, humectants, amphoteric surfactants, alcohols (more specifically, ethanol), compatible solutes, darkening agents, shine control agents, anti-microbial agents, anti-inflammatory agents, anti-mycotic agents, anti-parasite agents, external analgesics, photoprotectors, keratolytic agents, detergents, surfactants, moisturizers, nutrients, energy enhancers, anti-perspiration agents, astringents, deodorants, hair removers, firming agents, anti-callous agents, hair agent, nail agents, skin agents. Other active agents, especially cosmetically active agents that may be present include: hydroxy acids, alkylresorcinols, ascorbic acid & its derivatives, hyaluronic acid, retinoids, isosorbide dilinoleate, isosorbide disunflowerseedate, isosorbide dicaprylate, bakuchiol, D-panthenol, octyl methoxycinnamate, titanium dioxide, zinc oxides, octyl salicylate, homosalate, avobenzone, polyphenolics, carotenoids, free radical scavengers, ceramides, polyunsaturated fatty acids, essential fatty acids, enzymes, enzyme inhibitors, zingerone, acetyl zingerone, minerals, estrogens, steroids such as hydrocortisone, 2-dimethylaminoethanol, zinc salts, calcium salts, magnesium slats, coenzyme Q10, lipoic acid, amino acids, vitamins, acetyl-coenzyme A, niacin, riboflavin, thiamin, ribose, NADH, FADH2, Terminalia chebula fruit extract, Phyllanthus emblica fruit extract, Withania somnifera extract, aloe vera, feverfew, soy, and the like, as well as combinations thereof. In each instance, such other components are present in their typical amount: though, lesser amounts may be used where a synergy is found between the compounds of the present teaching and such other active agents. Especially preferred supplemental active ingredients include, but are not limited to acetyl zingerone, bakuchiol, isosorbide dicaprylate, Terminalia chebula fruit extract, ethyl linoleate, isosorbide dilinoleate, isosorbide disunflowerseedate and combinations of any two or more thereof. Each of these actives will be present in their traditional amounts, though, considering the common properties of certain of these actives and those of the compounds of the present teaching, it is believed that synergy exists whereby the amount of the conventional actives or the compounds of the present teaching or both may be reduced to achieve the same effect as found with the conventional active in the absence of the current compounds and vice-versa. Generally, though, these actives will be present in an amount of from about 0.01 to about 20 percent, preferably from about 0.1 to about 10 percent, more typically from about 0.1 to about 5 percent, by weight based on the total weight of the final composition.

Alternatively, or in addition thereto, these compositions also include other ingredients that have no or little bearing upon the intended end-use or application of the treatment aspect of these compositions, but aid in the preparation and/or longevity/stability of these compositions as well as the aesthetic aspects thereof. Exemplary inactive ingredients include solubilizers, surfactants, stabilizers, thickeners, preservatives, wetting agents, emulsifiers, suspending agents, dispersing agents, flavoring agents, fillers or extenders, disintegrating agents, absorption accelerators, absorbents, lubricants, buffers, dyes, perfumes, scents, opacifiers, colorants, etc, as well as combinations thereof; especially dermatologically acceptable amphoteric surfactants, ethanol and combinations of the two. Each of these is typically present, when present, in conventional amounts for those ingredients. It is not necessary to identify all of the possible specific active and non-active ingredients and additives that can be incorporated into the compositions of the present teaching as they are well known and widely available and, in any event, any attempt to do so would run on for page after page.

Another type of ingredient that may be used, one which is not really a skin active agent nor an inactive ingredient, are penetrants, Specifically, additives that, when applied to the skin, have a direct effect on the permeability of the skin barrier: increasing the speed with which and/or the amount by which certain other compounds penetrate the skin layers. Exemplary organic penetration enhancers include dimethyl sulfoxide; isopropyl myristate; decyl, undecyl or dodecyl alcohol; propylene glycol; polyethylene glycol; C₉₋₁₁, C₁₂₋₁₃ or C₁₂₋₁₅ fatty alcohols; azone; alkyl pyrrolidones; diethoxy glycol (Transcutol); lecithin; etc. Surfactants, especially amphoteric surfactants, can also be used as penetration enhancers. Again, each is employed in conventional amounts for such ingredients.

Alternatively, as noted above, the compounds of the present teaching may be incorporated into existing or newly formulated topical products of the like discussed above, including, especially, cosmetics, hair and skin care products, sunscreen products, moisturizers, acne treatment products, deodorants, and the like. In each instance, the amount and number of compounds according to the present teachings present in such products will be an effective amount for the objective sought, namely the purpose for adding the compounds.

The present teaching is also directed to the use of topical compositions in the treatment of individuals and animals. Specifically, the teaching is directed to methods of treating individuals and animals with the compounds the present teaching wherein the compounds are applied in a topical composition, with or without other active agents, to the skin, generally or a portion thereof, in an effective amount in order to rejuvenate and/or repair skin and its function, including, but not limited to, reducing the appearance of lines and/or wrinkles; reducing dermatological signs of chronological aging, photo-aging, hormonal aging, and/or actinic aging; reducing the noticeability of facial lines, wrinkles and age spots, facial wrinkles on the cheeks, forehead, wrinkles between the eyes, wrinkles above the eyes, and around the mouth, and particularly deep wrinkles or creases; reducing and/or diminishing the appearance and/or depth of lines and/or wrinkles: improving the appearance of suborbital lines and/or periorbital lines; reducing the appearance of crow's feet; rejuvenating and/or revitalizing skin, particularly aging skin; reducing skin fragility; reducing and/or treating hyperpigmentation or hypopigmentation, minimizing skin discoloration; improving skin tone, radiance, clarity and/or tautness; reducing and/or ameliorating skin sagging; improving skin firmness, plumpness, suppleness and/or softness; improving skin texture; improving skin barrier repair and/or function; improving the appearance of skin contours; restoring skin luster and/or brightness; minimizing dermatological signs of fatigue and/or stress; resisting environmental stress such as weather, sunburn, radiation damage, oxygen radical, hydrogen peroxide, and reactive oxygen intermediates; reducing skin cracking; replenishing ingredients in the skin decreased by aging and/or menopause; increasing cell proliferation and/or multiplication; increasing skin cell metabolism decreased by aging and/or menopause: improving skin moisturization; increasing skin elasticity and/or resiliency; enhancing exfoliation; improving microcirculation; reducing rosacea-associated skin redness; reducing skin erythema; reducing drug-induced skin atrophy; limiting cutaneous deformation; reducing progressive degradation of a dermal-epidermal junction and/or degradation of a cell-cell cohesion in skin; and any combinations thereof, Additionally, the compositions of the present teaching can be used as an adjuvant treatment following laser surgery or laser dermabrasion treatment to enhance aged and injured skin rejuvenation. Although the discussion herein is specific to skin related maladies and conditions, it is also to be noted that these topical compositions may be used as a convenient method to administer the compounds to the internal workings and components of the body for subsequent delivery to the target cells, tissue or organ: using the skin as simply a passageway or corridor for the compounds.

Again, as noted, these compounds and the topical compositions containing the same can be used for, but not limited to, treatment for aging skin and texture, minimizing fine lines and wrinkles, age spots, skin pigmentation and skin tone, dry skin, acne, psoriasis, and atopic dermatitis. The topical composition of this invention provides a visible improvement in skin condition of a subject shortly after application to the skin. Such prompt improvement involves coverage or masking of skin imperfections such as textural discontinuities (including those associated with skin aging, e.g., enlarged pores), or providing a more even skin tone or color. The compositions of the invention also provide visible improvements in skin condition following chronic topical application. “Chronic topical application” involves continued topical application of a composition over an extended period, preferably for a period of at least about one week, one month, three months, six months, or one year. Chronic regulation of skin condition involves improvement of skin condition following multiple topical applications.

Finally, again as noted above, the compounds of the present teaching as well as compositions comprising the same, alone or in combination with another skin active agent, particularly when present in a dermatologically or cosmetically acceptable carrier are for addressing and counteracting the ageing of the human skin, dry skin, pigment defects, barrier defects, UV damages on the skin, skin unevenness, such as wrinkles, fine lines, rough skin or large-pored skin, and diseases associated with skin ageing, such as defective keratinization, acne, eczema, inflammation, and skin atrophy.

While the foregoing is focused on skin, again, the utility of these compounds is not limited thereto and are applicable to any number of diseases and physiological conditions where activation/inhibitor of a number of enzymes, including, histone deacetylase (HDAC), COX-2, NF-κB, PGE-2 and IL-6, etc., maintenance/repair of stemness; elongation of telomeres; and boosting and regeneration of collagens; etc., are desired and beneficial.

In following, the present teaching also pertains to non-topical pharmaceutical compositions as well as oral supplements, especially nutritional supplements comprising at least one of the compounds according to Structure I and II wherein neither R² nor R¹ if present, is —H, most preferably those compounds of Structure II wherein one or, preferably, both R² and R³ are n-butyryl or isobutyryl, most especially n-butyryl, in combination with a pharmaceutically acceptable carrier or, in the case of supplements, the compounds are the active in combination with a carrier suitable for human consumption or, more likely, are additional active ingredient to a conventional supplement, particularly nutritional supplement, e.g., tablets, capsules, gummies, and powders as well as drinks, especially hydration drinks, and energy bars. The compounds of the present teaching are present in an effective amount for achieving the desired/intended outcome for which the compound(s) is(are) employed, e.g., anti-inflammatory, metal chelation therapy, gastrointestinal regulation, reduced oxidative stress in internal organs and physiological functions and processes and other typical applications for butyrates and zinc supplements. In all cases the composition will contain an effective amount of the compounds; a therapeutically effective amount in the case of pharmaceutical compositions. Generally, an effective amount in both instances is from about 0.01 to about 20 percent, preferably from about 0.1 to about 10 percent, more typically from about 0.1 to about 5 percent, by weight based on the total weight of the final composition.

The form of the non-topical treatment composition and mode of administration depends upon the specific nature of the malady and/or disease and/or condition of symptom to be addressed. Indeed, the specific mode of application or administration is, in part, dependent upon the form of the non-topical composition, the primary purpose or target of its application, e.g., the application may be oral if intending to address the disease generally or provide the compound as or as an additional nutrient. The non-topical formulation containing the compounds of the present teaching may be in the form of a solid, gel, paste, liquid, suspension, dispersion, subdermal patch, suppository, liposome formation, mouth wash, enema, injectable solution, eye drop, ear drop, drip infusion, microcapsule, etc. Suitable modes of administration include, for example, intradermal, intramuscular, intraperitoneal, intravenous, subcutaneous, intranasal, epidural, oral, sublingual, intracerebral, intravaginal, transdermal, rectal, or inhalation. The preferred mode is oral and those methods that involve absorption through epithelial or mucous linings (e.g., oral mucosa, rectal, and intestinal mucosa, etc.), preferably oral. Furthermore, depending in part upon the form and primary purpose or target of the administration, the non-pharmaceutical compositions of the present disclosure may be administered systemically or locally. Finally, the form of the non-pharmaceutical composition and its delivery system may also vary depending upon the parameters already noted. For example, orally administered pharmaceutical compositions of the present teaching may be in encapsulated form, e.g., encapsulated in liposomes, or as microparticles, microcapsules, capsules, etc. Another factor for consideration of the non-topical applications is the concept of dosing. These compositions will be administered in a therapeutically effective does: said dosage being calculated based on the concentration of the active compound, the weight and age of the individual to whom it is to be administered, the mode of administration, and other factors that may affect metabolism of the active ingredient, etc.: all of which is within the purview of one of ordinary skill in the art.

Finally, the present teaching is also directed to the treatment of individual suffering from various maladies and disease conditions with a focus on eradicating or mediating the symptoms thereof and/or improving the normal function of the body. In this respect, the compounds and composition of the present teaching have many attributes in activating/inhibiting various enzymes and gene expressions which are associated with various medical conditions including inflammation and maladies associated with oxidative stress of internal organs and physiological functions and processes, as well as utility in metal chelation therapy, cancer treatment, treatment of gastrointestinal disorders and regulation, etc. Hence, the present teaching is also directed to the use of the non-topical compositions in the treatment of individuals and animals and in improving overall health through supplements. Specifically, the teaching is directed to methods of treating individuals and animals with the compounds the present teaching as well as a method for enhancing overall health and wellness by their use as nutritional supplements.

Having characterized and described the present teachings, attention is now directed to the following examples pertaining to the preparation of the compounds of the present invention as well as demonstrating the efficacy thereof, particularly in relation to their impact on HDAC, skin hydration, COX-2, and other skin related processes and gene expressions.

### EXAMPLES

### Example 1: Preparation Di-n-butyryl Lysine

In tetrahydrofuran and distilled water, L-Lysine HCl (1 mole) and butyric anhydride (2.2 moles) or butyric acid chloride (2.2 moles) were added and stirred for 12 to 16 hours at 30±5° C. Then sodium carbonate was added to the reaction mixture and stirred for another 12 to 16 hours at 30±5° C. Following that, sodium chloride and hydrochloric acid were added and the mixture stirred for 30 min following which the aqueous layer was allowed to separate and was then separated. The remaining organic layer was subjected to distillation under vacuum at <40° C. The crude product was further purified by column chromatography. The purified product was characterized to be di-n-butyryl lysine (also referred to as di-n-butyroyl lysine) by spectroscopic methods.

### Example 2: Preparation Di-isobutyryl Lysine

Di-isobutyryl lysinate (also referred to as di-isobutyroyl lysine) is prepared according to the same procedure as set forth in Example 1 with the exception that isobutyric anhydride (2.2 moles) or isobutyric acid chloride (2.2 moles) is substituted for the butyric anhydride or butyric acid chloride, respectively.

### Example 3: Preparation n-butyryl Lysine

n-butyryl Lysine (also referred to as n-butyroyl lysine) is prepared according to the same procedure as set forth in Example 1 with the exception that amount of n-butyric anhydride or n-butyric acid chloride is halved (1.1 moles).

### Example 4: Preparation isobutyryl Lysine

Isobutyryl lysine (also referred to as isobutyroyl lysine) is prepared according to the same procedure as set forth in Example 2 with the exception that amount of isobutyric acid chloride is halved (1.1 moles).

### Example 5: Preparation of n-butyryl leucinate

Leucine HCl (1 mole) and butyric acid chloride (1.1 moles) were added to a solution of tetrahydrofuran and distilled water and the mixture stirred for 12 to 16 hours at 30±5° C. Then sodium carbonate was added to the reaction mixture and the mixture stirred for another 12 to 16 hours at 30±5° C. Following that, sodium chloride and hydrochloric acid were added and the mixture stirred for 30 min following which the aqueous layer was allowed to separate before being separated. The remaining organic layer was subjected to distillation under vacuum at <40° C. and the crude product further purified by column chromatography. The purified product was dried under vacuum at <40° C. and identified by spectral analysis as n-butyryl leucinate (also referred to as n-butyroyl leucinate).

### Example 6: Preparation of Zinc Di-(di-n-butyryl Lysinate)

Di-n-butyryl lysine (2 moles) from Example 1 was mixed with methanol and zinc hydroxide (1.1 moles) and heated to 65° C. and the mixture maintained at that temperature for 12 hours. The reaction mixture was then filtered to remove any undissolved salt. The filtrate was subjected to distillation to remove the solvent under vacuum at <40° C., The crude product was washed with acetone and n-heptane. The purified product was dried under vacuum at <40° C. and identified by spectral analysis as zinc di-(di-n-butyryl lysinate) (also referred to as zinc di-(di-n-butyroyl lysinate).

### Example 7: Preparation of Zinc Di-(di-isobutyryl Lysinate)

Zinc di-(di-isobutyryl lysinate) is prepared according to the same procedure as set forth in Example 6 with the exception that di-isobutyryl lysine of Example 2 is substituted for di-n-butyryl lysine.

### Example 8: Preparation of Zinc Di-(n-butyryl lysinate) and Zinc Di-(isobutyryl lysinate)

Zinc di-(n-butyryl lysinate) and Zinc di-(isobutyryl lysinate) are prepared in accordance with the process of Example 6 with the exception that n-butyryl lysine and isobutyryl lysine of Examples 3 and 4, respectively, are substituted for the di-n-butyryl lysine and di-n-butyryl lysine, respectively.

### Example 9: Preparation of Calcium Di-(di-n-butyryl Lysinate)

Di-n-butyryl lysine (2 moles) from Example 1 was mixed with methanol and Calcium hydroxide (1.1 moles) and heated to 65° C. and the mixture maintained at that temperature for 12 hours. The reaction mixture was then filtered to remove any undissolved salt. The filtrate was subjected to distillation to remove the solvent under vacuum at <40° C. The crude product was washed with acetone and n-heptane. The purified product was dried under vacuum at <40° C. and identified by spectral analysis as calcium di-(di-n-butyryl lysinate).

### Example 10: Preparation of Calcium Di-(di-isobutyryl Lysinate)

Calcium di-(di-isobutyryl lysinate) is prepared according the same procedure as set forth in Example 9 with the exception that di-isobutyryl lysine of Example 2 is substituted for the di-n-butyryl lysine.

### Example 11: Process for Making Zinc Di-Lysinate

Zinc di-lysinate was prepared in accordance with the process of Example 6 with the exception that lysine HCl was substituted for the di-n-butyryl lysine.

### Example 12: Process for Making Zinc Di-(n-Butyryl Leucinate)

Zinc di-(n-butyryl leucinate) was made in accordance with the process of Example 6 with the exception that n-butyryl leucine was substituted for di-n-butyryl lysine.

### Example 13: Process for Making Zinc Di-(n-Butyryl Isoleucinate)

Zinc di-(n-Butyryl isoleucinate) was made in accordance with the process of Example 6 with the exception that n-butyryl isoleucine was substituted for di-n-butyryl lysine.

### Example 14: Process for Making Zinc Di-(Butyryl Valinate)

Zinc di-(n-butyryl valinate) was made in accordance with the process of Example 6 with the exception that n-butyryl valine was substituted for di-n-butyryl lysine.

### Example 15: Process for Making the Magnesium Salts

The process for making magnesium salts comprises the following steps; (1) adding 75-200 g of the modified amino acids and 100-400 g deionized water into a four-mouth reaction flask and stirring to dissolve the amino acid, (2) adding 20-53 g of magnesium oxide to the flask and heating the mixture to about 70-80° C. stirring reaction mix for 2-3 hours, (3) opening vacuum, and stirring at 70-80° C. for concentration for 2-3 hours, (4) after concentration, the temperature is lowered to 45-50° C., and 90-250 g of alcohol is added and the mix stirred and allowed to cool to the room temperature, and (5) performing suction filtration with a Buchner funnel, and vacuum drying at 40° C. for 2 hours to obtain the magnesium salts.

### Example 16: Process for Making the Manganese Salts

The process for making manganese salts comprises the same process as in Example 15 with the exception that manganese oxide is substituted for the magnesium oxide.

### Example 17: HDAC Inhibitory Activity

### General Procedure

Test materials were stored at room temperature and stock solutions were prepared at 20 mg/ml in DMSO. Further dilutions were made in sterile distilled water immediately before being added to the reaction mixture containing a substrate, and the reaction was triggered by the addition of purified HDAC enzymes, Double distilled water was the negative control and trichostatin (1.5 ug/ml) was used as a positive control for validation of each study. The test procedure was as directed in the assay kits. The products of the reactions were measured by fluorometry at ex/em 350/450 nm using multi-well plate fluorometer Cytofluor 4000 (Applied Biosystem, Foster City, CA) or using SpectraMax i3× Multi-Mode Detection Platform from Molecular Devices (Sunnyvale, CA).

Statistical significance was assessed with a two-tail Student test. Deviations of ≤20% as compared to water control with p values below 0.05 were considered statistically significant.

HDAC Enzymes used for demonstrating the influence/effectiveness of the compounds of the present teaching on HDAC expression.
HDAC 1 fluorogenic assay kit #50061 used, BPS Biosciences, San Diego, CA
HDAC 2 fluorogenic assay kit #50062 used, BPS Biosciences, San Diego, CA
HDAC 3 fluorogenic assay kit #50073 used, BPS Biosciences, San Diego, CA
HDAC 4 fluorogenic assay kit #50064 used, BPS Biosciences, San Diego, CA
HDAC 5 fluorogenic assay kit #50065 used, BPS Biosciences, San Diego, CA
HDAC 6 fluorogenic assay kit #50076 used, BPS Biosciences, San Diego, CA
HDAC 7 fluorogenic assay kit #50067 used, BPS Biosciences, San Diego, CA
HDAC 8 fluorogenic assay kit #50068 used, BPS Biosciences, San Diego, CA
HDAC 9 fluorogenic assay kit #50069 used, BPS Biosciences, San Diego, CA
HDAC 10 fluorogenic assay kit #50060 used, BPS Biosciences, San Diego, CA
HDAC 11 fluorogenic assay kit #50687 used, BPS Biosciences, San Diego, CA

Tables 1A through 1C present the results of the inhibitory/excitatory activity of compounds of the present teaching versus various controls on select HDACs. In each table the values present the remaining activity level: hence, an activity level of 23% indicates that there is 77% inhibition.

TABLE 1A
HDAC enzymatic activity expressed as % of water-treated controls.

| | | Products | | | | | | | |
|---|---|---|---|---|---|---|---|---|---|
| | | HDAC 1 | | HDAC 2 | | HDAC 3 | | HDAC 4 | |
| | | activ- | p- | activ- | p- | activ- | p- | activ- | p- |
| | | ity | value | ity | value | ity | value | ity | value |
| Zn di-(di-n-butyryl Lysinate) | | | | | | | | | |
| 200 | ug/ml | 45 | 0.000 | 15 | 0.000 | 4 | 0.000 | | |
| 100 | ug/ml | 49 | 0.000 | 17 | 0.000 | | | 39 | 0.000 |
| 50 | ug/ml | 38 | 0.000 | 20 | 0.000 | 5 | 0.000 | 60 | 0.000 |
| 25 | ug/ml | 32 | 0.000 | 20 | 0.000 | 14 | 0.000 | 120 | 0.025 |
| 5 | ug/ml | 30 | 0.000 | | | | | | |
| Zn dilysinate | | | | | | | | | |
| 200 | ug/ml | | | 9 | 0.000 | 5 | 0.000 | | |
| 100 | ug/ml | 32 | 0.014 | 1 | 0.000 | | | 63 | 0.000 |
| 50 | ug/ml | 44 | 0.038 | 1 | 0.000 | 8 | 0.000 | 92 | 0.130 |
| 25 | ug/ml | 52 | 0.104 | 12 | 0.000 | | | 100 | 1.000 |
| 10 | ug/ml | | | | | 18 | 0.000 | | |
| Ca di-(di-n-butyryl lysinate) | | | | | | | | | |
| 200 | ug/ml | | | 94 | 0.016 | 119 | 0.048 | | |
| 100 | ug/ml | 135 | 0.267 | 97 | 0.157 | 121 | 0.029 | | |
| 50 | ug/ml | 89 | 0.688 | 90 | 0.001 | 120 | 0.047 | | |
| 25 | ug/ml | 80 | 0.433 | 96 | 0.066 | 116 | 0.076 | | |

Ca dilysinate

| | | | |
|---|---|---|---|
| 100 | ug/ml | 107 | 0.175 |
| 50 | ug/ml | 101 | 0.761 |
| 25 | ug/ml | 109 | 0.164 |

Ca n-butyrate

| 100 | ug/ml | | | | | 109 | 0.067 |
|---|---|---|---|---|---|---|---|
| 50 | ug/ml | | | | | 102 | 0.659 |
| 25 | ug/ml | | | | | 104 | 0.542 |
| Lysine | | | | | | | |
| 100 | ug/ml | 93 | 0.699 | | | 97 | 0.498 |
| 50 | ug/ml | | | | | 100 | 0.995 |
| 25 | ug/ml | | | | | 98 | 0.739 |
| 20 | ug/ml | 90 | 0.510 | | | | |
| Mg di-(di-n-butyryl lysinate) | | | | | | | |
| 200 | ug/ml | 74 | 0.436 | 117 | 0.193 | | |
| 100 | ug/ml | | | 114 | 0.239 | | |

Zn di-(n-butyryl leucinate)

| 200 | ug/ml | | | 28 | 0.000 | | | | |
|---|---|---|---|---|---|---|---|---|---|
| 100 | ug/ml | | | 31 | 0.000 | | | | |
| 50 | ug/ml | | | 37 | 0.000 | | | | |
| 25 | ug/ml | | | 39 | 0.000 | | | | |
| Sodium butyrate (positive control) | | | | | | | | | |
| 400 | ug/ml | 38 | 0.015 | 95 | 0.046 | 49 | 0.129 | | |
| 200 | ug/ml | 39 | 0.017 | 94 | 0.022 | 60 | 0.264 | | |
| 100 | ug/ml | 46 | 0.027 | 93 | 0.007 | 82 | 0.841 | 96 | .0390 |
| 50 | ug/ml | 109 | 0.778 | 89 | 0.000 | 98 | 0.601 | 96 | 0.381 |
| 25 | ug/ml | | | | | | | 93 | 0.272 |
| Lysine | | | | | | | | | |
| 100 | ug/ml | 93 | 0.699 | | | | | | |
| 20 | ug/ml | 90 | 0.510 | | | | | | |

TABLE 1B
HDAC enzymatic activity expressed as % of water-treated controls.

| | | Products | | | | | | | |
|---|---|---|---|---|---|---|---|---|---|
| | | HDAC 5 | | HDAC 6 | | HDAC 7 | | HDAC 8 | |
| | | activ- | p- | activ- | p- | activ- | p- | activ- | p- |
| | | ity | value | ity | value | ity | value | ity | value |
| Zn di-(di-n-butyryl lysinate) | | | | | | | | | |
| 200 | ug/ml | 17 | 0.001 | 19 | 0.000 | 9 | 0.000 | 17 | 0.001 |
| 100 | ug/ml | 33 | 0.005 | 21 | 0.000 | 9 | 0.000 | 13 | 0.001 |
| 50 | ug/ml | 65 | 0.082 | 19 | 0.000 | 15 | 0.000 | 15 | 0.001 |
| 25 | ug/ml | 89 | 0.532 | 18 | 0.000 | 28 | 0.000 | 15 | 0.001 |
| 5 | ug/ml | | | 25 | 0.000 | 56 | 0.000 | 15 | 0.001 |
| Zn dilysinate | | | | | | | | | |
| 200 | ug/ml | 14 | 0.001 | 8 | 0.000 | 10 | 0.000 | 12 | 0.001 |
| 100 | ug/ml | 31 | 0.004 | 14 | 0.000 | 10 | 0.000 | 13 | 0.001 |
| 50 | ug/ml | 52 | 0.026 | 17 | 0.000 | 18 | 0.000 | 14 | 0.001 |
| 25 | ug/ml | 73 | 0.158 | 16 | 0.000 | 30 | 0.000 | 13 | 0.001 |
| 5 | ug/ml | | | 22 | 0.000 | | | 13 | 0.001 |
| Ca di-(di-n-butyryl lysinate) | | | | | | | | | |
| 200 | ug/ml | 88 | 0.510 | 134 | 0.010 | 97 | 0.507 | 111 | 0.588 |
| 100 | ug/ml | 117 | .0379 | 128 | 0.015 | 99 | 0.880 | 155 | 0.017 |
| 50 | ug/ml | 123 | 0.251 | 124 | 0.031 | 99 | 0.851 | 174 | 0.003 |
| 25 | ug/ml | 128 | .0167 | 97 | 0.702 | 101 | 0.872 | 148 | 0.032 |
| 5 | ug/ml | 144 | 0.035 | 105 | 0.708 | | | 91 | 0.673 |

Zn di-(n-butyryl leucinate)

| 200 | ug/ml | 26 | 0.003 | 17 | 0.000 | 9 | 0.000 | 14 | 0.001 |
|---|---|---|---|---|---|---|---|---|---|
| 100 | ug/ml | 50 | 0.021 | 20 | 0.000 | 9 | 0.000 | 16 | 0.001 |
| 50 | ug/ml | 91 | 0.627 | 21 | 0.000 | 11 | 0.000 | 16 | 0.001 |
| 25 | ug/ml | 128 | 0.146 | 20 | 0.000 | 18 | 0.000 | 16 | 0.001 |
| 5 | ug/ml | | | 20 | 0.000 | 41 | 0.000 | 7 | 0.001 |
| Mg di-(di-n-butyryl lysinate) | | | | | | | | | |
| 200 | ug/ml | | | | | 100 | 0.954 | | |
| 100 | ug/ml | | | | | 100 | 0.976 | | |
| Sodium butyrate (positive control) | | | | | | | | | |
| 400 | ug/ml | 11 | 0.001 | 102 | 0.887 | 91 | 0.099 | 14 | 0.001 |
| 200 | ug/ml | 15 | 0.001 | 92 | 0.565 | 87 | 0.280 | 20 | 0.002 |
| 100 | ug/ml | 26 | 0.003 | 110 | 0.334 | 94 | 0.247 | 20 | 0.002 |
| 50 | ug/ml | 38 | 0.008 | 107 | 0.549 | 96 | 0.493 | 25 | 0.002 |

TABLE 1C
HDAC enzymatic activity expressed as % of water-treated controls.

| | | Products | | | | | |
|---|---|---|---|---|---|---|---|
| | | HDAC 9 | | HDAC 10 | | HDAC 11 | |
| | | | p- | | p- | | p- |
| | | activity | value | activity | value | activity | value |
| Zn di-(di-n-butyryl lysinate) | | | | | | | |
| 200 | ug/ml | 20 | 0.000 | 7 | 0.000 | 43 | 0.0000 |
| 100 | ug/ml | 19 | 0.000 | 9 | 0.000 | 22 | 0.000 |
| 50 | ug/ml | 25 | 0.001 | 13 | 0.000 | 17 | 0.000 |
| 25 | ug/ml | 29 | 0.001 | 15 | 0.000 | 63 | 0.016 |
| 5 | ug/ml | 39 | 0.004 | 21 | 0.000 | 102 | 0.807 |
| Zn dilysinate | | | | | | | |
| 200 | ug/ml | 16 | 0.000 | 6 | 0.000 | 38 | 0.000 |
| 100 | ug/ml | 15 | 0.000 | 9 | 0.000 | 47 | 0.000 |
| 50 | ug/ml | 15 | 0.000 | 12 | 0.000 | 84 | 0.050 |
| 25 | ug/ml | 18 | 0.000 | 19 | 0.016 | 91 | 0.245 |
| 5 | ug/ml | 21 | 0.001 | | | | |
| Ca di-(di-n-butyryl lysinate) | | | | | | | |
| 200 | ug/ml | 258 | 0.000 | 90 | 0.154 | 97 | 0.670 |
| 100 | ug/ml | 289 | 0.000 | 104 | 0.606 | 101 | 0.910 |
| 50 | ug/ml | 242 | 0.000 | 103 | 0.713 | 100 | 0.957 |
| 25 | ug/ml | 178 | 0.000 | 105 | 0.453 | 106 | 0.446 |
| 5 | ug/ml | 212 | 0.000 | | | | |

Zn di-(n-butyryl leucinate)

| 200 | ug/ml | 22 | 0.001 | 6 | 0.000 | 39 | 0.000 |
|---|---|---|---|---|---|---|---|
| 100 | ug/ml | 30 | 0.001 | 8 | 0.000 | 71 | 0.000 |
| 50 | ug/ml | 44 | 0.008 | 10 | 0.000 | 93 | 0.826 |
| 25 | ug/ml | 49 | 0.011 | 13 | 0.000 | 100 | 0.982 |
| 5 | ug/ml | 15 | 0.000 | 21 | 0.000 | 102 | 0.807 |
| Mg di-(di-n-butyryl lysinate) | | | | | | | |
| 200 | ug/ml | | | 102 | 0.808 | 91 | 0.256 |
| 100 | ug/ml | | | 98 | 0.743 | 91 | 0.266 |
| Sodium butyrate (positive control) | | | | | | | |
| 400 | ug/ml | 58 | 0.029 | 61 | 0.000 | 91 | 0.51 |
| 200 | ug/ml | 57 | 0.026 | 93 | 0.297 | 84 | 0.044 |
| 100 | ug/ml | 53 | 0.016 | 103 | 0.718 | 87 | 0.124 |
| 50 | ug/ml | 55 | 0.020 | 100 | 0.964 | 89 | 0.165 |

Zn di-(di-n-butyryl lysinate) + Zn dilysinate (1:1 w/w)

| | | |
|---|---|---|
| 100 ug/ml + 100 ug | 68 | 0.023 |
| 50 ug/ml + 50 ug/ml | 61 | 0.039 |
| 25 ug/ml + 25 ug | 40 | 0.001 |

### Example 18: Reprograming of Aged Mesenchymal Stem Cells (MSCs) [0000]

MSCs are a multipotent type of stem cell that, directly or indirectly, have a significant impact on a number of physiological processes, including the proliferation and differentiation of epidermal cells and regeneration of the epidermal layer; however, as they age they show lower stemness and regenerative potential. MSCs are shown to enhance wound healing, to restore the barrier function of the skin, and to address skin aging, particularly aging due to sun exposure as well as natural senescence, including reducing the manifestation and/or degree of wrinkles. An evaluation was conducted to ascertain the effect, if any, of zinc di-(di-n-butyryl lysinate) (“ZDBL”) on MSCs. Specifically, normal human adipose-derived mesenchymal stem cells (MSC) (ATCC, Manassas, VA—PCS-500-011) were seeded in mesenchymal stem cell basal medium (ATCC, Manassas, VA—PCS500-030) supplemented with the MSC growth kit starvation media (ATCC, Manassas, VA—PCS500-040-811S) and cultured at 37° C. in a humidified 5% CO₂ atmosphere using an VWR tissue culture incubator, Approximately 24 hours after a portion of the cells was placed in the starvation medium to generate stress, they were exposed to the ZDBL (10 ug/ml) and incubated for 72 hours: the remaining portion was not exposed to the ZDBL. RNA was then extracted for gene expression quantification by qPCR. Table 2 presents the results of the PCR analysis (fold change of ZDBL versus control), which showed an up-regulation of 2 out of 3 Yamanaka factors as well as other genes associated with reversing or reducing the effects of biological aging of the skin.

TABLE 2
Gene Expression Profile

| Gene | Fold | |
|---|---|---|
| Symbol | Change | Gene Description |
| BMP7 | 1.8* | HDAC inhibitors, such as |
| | | Trichostatin A inhibit epithelial-to- |
| | | mesenchymal transition (EMT) |
| | | through upregulation of BMP7 |
| | | (PMID: 17135397) |
| DACT1 | 2.2** | DACT1 is important for embryonic |
| | | feeder cells to support the |
| | | proliferation of human epidermal |
| | | keratinocytes (PMC4640957). |
| DLK1 | 2.4** | A non-canonical NOTCH ligand |
| | | supporting stemness |
| DPP4 | −1.8** | DPP4 inhibition improves hair |
| | | follicle activation and regeneration |
| | | (PMID: 37236597) |
| SQSTM1/p62 | 1.7* | It is involved in autophagy, |
| | | and its loss has been linked to |
| | | accelerated aging and to age-related |
| | | pathologies (PMID: 24557832). |
| OCT4 | 1.7** | It is one of the Yamanaka factors⁺, |
| | | and supports stemness (PMID: |
| | | 18423197; PMID: 18276851) |
| SOX2 | 2.4* | One of the three Yamanaka |
| | | factors⁺, it promotes stemness and |
| | | suppresses aging. NANOG, SOX2 |
| | | and OCT4 represent the core |
| | | regulatory network that |
| | | suppresses differentiation-associated |
| | | genes, maintaining the pluripotency |
| | | of mesenchymal stem cells |
| | | (PMID: 31130693, PMID: |
| | | 18423197; PMID: 18276851) |
| TP63 | 3.0** | Supports stem cell maintenance |
| | | and self-renewal (stemness; |
| | | PMC10377110) |
| WNT2 | 1.9** | Wnt signaling is triggered by |
| | | injury to the skin and promotes |
| | | activation of stem cells and wound |
| | | healing. WNT2 is a key signaling |
| | | protein in stem cells (PMID: 20005973) |

Significant (p = ≤0.003);
**Highly significant (p = ≤0.001)
⁺Reference: Takashi & Yamanaka, Nat Rev Mol Cell Biol, 17: 183-93, 2016

### Example 19: Telomerase Activation

Telomeres are protective caps on the ends of chromosomes which shorten with each cell division. Progressive shortening of the telomeres leads to senescence, apoptosis, or oncogenic transformation of somatic cells, affecting the health and lifespan of an individual: shorter telomeres have been associated with increased incidence of diseases and poor survival, Telomerase is an enzyme which adds nucleotide sequences back to the telomere. An investigation was conducted to ascertain the effect, if any, of the compounds of the present teaching on telomerase activity.

Normal neonatal human dermal fibroblasts (HDF cat, #2310; ScienCell, Oceanside, CA) were grown to confluence in the 6 well plate format and incubated with the test substances identified in Table 3 for three days. Following incubation, the cells were lysed and telomerase activity was determined with Telomerase Activity Quantification qPCR Assay Kit from ScienCell (cat. #8928). Forget-Me-Not™ EvaGreen® qPCR Master Mix (Low ROX) (Biotium, Fremont, CA) was used for real-time quantitative PCR on C1000 Touch System with CFX384™ optical module Detection. For quantifying the level of activity, the Comparative Cq method was used, following the equation:

$$Δ {Cq}_{( TPS )} = {Cq}_{( TPS , H 2 O )} - {Cq}_{( TPS , Sample )}$$

where TPS is telomere primer set, which recognizes and amplifies newly synthesized telomere sequences and Cq_{(TPS)} is the Quantification Cycle Value obtained from the qPCR software. The relative telomerase activity difference between the experimental condition and control is expressed as 2^{-ΔCq} Telomerase activity was considered different if the level of expression was reasonably high (Cq≤33 cycles to detect), the difference vs. control was >2 fold and the p value, assessed with two-tailed Student test, was <0.05. The results are summarized in Table 3.

### Example 20: Boosting of Collagen

Collagen makes up 70-80% of the dry weight of the skin, with Collagen 1 being the main collagen, representing 80%-90% of skin collagen, and primarily responsible for the mechanical and structural integrity of the skin. Collagen IV, another key skin collagen, is the primary collagen found in the extracellular basement membranes, particularly the lamina densa, separating a variety of epithelial and endothelial cells. There are six human genes associated with collagen IV, specifically COL4A1, COL4A2, COL4A3, COL4A4, COL4A5 and COL4A5 (A M Abreu-Velez and M S Howard, Collagen IV in Normal Skin and in Pathological Processes, N Am J Med Sci, 4(1): 1-8, 2012). In recent years, the basement membrane has been recognized as an important regulator of cell behavior, rather than just a structural feature of tissues.

TABLE 3
Quantification of telomerase activity, relative telomerase
activity vs. water and fold change (FC) vs. water.

| | | ΔCq | | Telomerase |
|---|---|---|---|---|
| | Average | sample- | p | Activity (FC |
| Tested Substance | Cq_{(TPS)} | water | value | vs. water) |
| H₂O | 33.8 | 0.0 | 1.000 | 1.0 |
| ZnDBL 50 μg/ml | 30.0 | 3.9 | 0.044 | 14.6 |
| ZnDBL 10 μg/ml | 30.7 | 3.2 | 0.001 | 8.9 |
| Trichostatin-A 300 nM | 32.3 | 1.5 | 0.000 | 2.8 |
| Positive control) | | | | |
| Na Butyrate 100 μg/ml | 33.7 | 0.1 | 0.715 | 1.1 |
| Na Butyrate 50 μg/ml | 34.3 | −0.5 | 0.722 | 0.7 |
| Telomerase Positive Control | 22.4 | N/A | N/A | N/A |
| Telomerase Negative Control | 38.6 | 0.037 | N/A | N/A |
| No Telomerase Negative | 30.5 | N/A | N/A | N/A |
| Control (H₂0 RNase free | | | | |
| from kit) | | | | |

Cq_{(TPS)}: Quantification Cycle Value, N/A: does not apply.

From early adulthood, fibroblasts become less active and collagen production declines by about 1.0%-1.5% a year. This decrease in collagen is one of the characteristic hallmarks associated with the appearance of fine lines and deeper wrinkles during the ageing process. An investigation was conducted to ascertain the effect, if any, of the compounds of the present teaching on collagen activation.

In the study, human neonatal fibroblasts were seeded into the individual wells of a 48-well plate in 0.25 ml of Fibroblasts Growth Media (FGM) and incubated overnight at 37±2° C. and a 5±1% CO₂ atmosphere. On the following day the media was removed via aspiration to eliminate non-adherent cells and replaced with 0.5 ml of fresh FGM. The cells were grown until confluent, with a media change every 48 to 72 hours. Upon reaching confluency the cells were treated for 24 hours with Dulbecco's Modified Eagle's medium (DMED) supplemented with 1.5% Fetal Bovine Serum (FBS) to wash out any effects from the growth factors included in the normal culture media. After this 24-hour wash out period the cells were treated with the test materials at the specified concentrations dissolved in FGM with 1.5% FBS. Sodium ascorbate (10 ug/ml) was used as a positive control. Untreated cells (negative controls) only received DMEM with 1% FBS, The cells were incubated for 48 hours and at the end of the incubation period, cell culture medium was collected and either stored frozen (−75 C) or assayed immediately. Materials were tested in triplicate.

### Collagen I Assay

A series of type I collagen peptide standards was prepared ranging from 0 ng/ml to 640 ng/ml. An ELISA microplate was prepared by removing any unneeded strips from the plate frame followed by the addition of 100 μl of peroxidase labelled anti-procollagen type I-C peptide antibody to each well used in the assay. A 20 μl sample of each of the collected tissue culture media or a standard was then added to appropriate wells and the microplate covered and allowed to incubate for 3±0.25 hours at 37 C. Following incubation, the wells were aspirated and washed three times with 400 μl of wash buffer. After the last wash was remove 100 μl of peroxidase substrate solution (hydrogen peroxide+tetramethylbenzidine as a chromogen) was added to each well and the plate incubated for 15±4 minutes at room temperature. Following this incubation, 100 μl of stop solution (1 N sulfuric acid) was added to each well and the plate read using a microplate reader at 450 nm. The results are presented in Table 4.

### Collagen IV Assay

A series of type IV collagen peptide standards was prepared ranging from 0 ng/ml to 640 ng/ml. 100 μl of each standard or of each of the collected tissue culture media were added to the wells of type IV collagen ELISA plates. The plates were then incubated at 37° C. for 1 hour. After incubation, the ELISA plates were then washed twice with wash buffer, followed by the addition of 100 μl of detection antibody solution. The ELISA plates were then incubated for 1 hour at 37° C. Following incubation, the ELISA plates were washed with wash solution followed by the addition of 100 μl HEP conjugate solution and incubated at 37° C. for 30 minutes. Following the latter incubation, the ELISA plates were again washed and 100 μl of the substrate solution was added to each well and the well plates incubated for 10-30 minutes at room temperature to allow the color generation reaction to occur. At the end of the color generation reaction 100 μl of stop solution (1 N sulfuric acid) was added to each well and the plates read at 460 nm using a plate reader. The results are presented in Table 6.

### Calculation/Statistical Analysis

To quantify the amount of each substance present, a standard curve was generated using known concentrations of each substance. A regression analysis was performed to establish the line that best fit those data points. Absorbance values for the test materials and untreated samples were used to estimate the amount of each substance present in each sample. Treatment means were compared using ANOVA, with an n+3 per treatment. Statistical significance was set at p≤0.05.

TABLE 4
Type I Collagen Assay

| Treatment | C-Peptide (ng/ml) |
|---|---|
| Untreated | 1255 ± 83 |
| 0.1% DMSO (control) | 1317 ± 65 |
| 10 μg/ml Na ascorbate (control) | 4018 ± 196* |
| 30 μg/ml Zn di-(di-n-butyryl lysinate) | 1958 ± 125* |
| 20 μg/ml Zn di-(di-n-butyryl lysinate) | 1541 ± 94 |
| 10 μg/ml Zn di-(di-n-butyryl lysinate) | 1328 ± 48 |
| 100 μg/ml Na butyrate | 1277 ± 216 |
| 60 μg/ml Na butyrate | 1196 ± 175 |
| 30 μg/ml Na butyrate | 1262 ± 95 |
| 10 μg/ml Na butyrate | 1445 ± 168 |

*Statistically significant difference between treated vs untreated (p ≤ 0.05)

As evident from Table 4, zinc di-(di-n-butyryllysinate) at concentrations of 20 μg/ml and 30 ug/ml demonstrated a statistically significant (p<0.05) boosting of Collagen I: ˜50% boost vs control at 30 μg/ml. Likewise, as evident from Table 5, zinc di-(di-n-butyryl lysinate) manifested a dose dependent and statistically significant (p<0.05) boosting of Collagen IV: ˜320% and ˜250% versus control at 30 μg/ml and 20 μg/ml, respectively. Surprisingly, Na butyrate didn't show any Collagen I or Collagen IV boosting activity, even with concentrations as high as 100 μg/ml.

TABLE 5
Type IV Collagen Assay

| Treatment | C-Peptide (ng/ml) |
|---|---|
| Untreated | 7.76 ± 0.92 |
| 0.1% DMSO | 8.37 ± 1.15 |
| 10 μg/ml Na ascorbate | 67.70 ± 5.03* |
| 30 μg/ml Zn di-(di-n-butyryl lysinate) | 26.98 ± 1.37* |
| 20 μg/ml Zn di-(di-n-butyryl lysinate) | 21.19 ± 1.47* |
| 10 μg/ml Zn di-(di-n-butyryl lysinate) | 13.41 ± 1.40 |
| 100 μg/ml Na butyrate | 12.80 ± 1.83 |
| 60 μg/ml Na butyrate | 11.88 ± 1.37 |
| 30 μg/ml Na butyrate | 7.61 ± 0.70 |
| 10 μg/ml Na butyrate | 9.29 ± 0.70 |

*Statistically significant difference between treated vs untreated (p ≤ 0.05)

### Example 21: Prolidase Activation

Prolidase (PEPD) is a cytosolic imidodipeptidase that cleaves di- and tripeptides containing carboxyl-terminal proline or hydroxyproline and is essential in protein metabolism, collagen turnover and regeneration, and matrix remodeling. Its activity is regulated by a number of mechanisms including activation of the β₁-integrin receptor, insulin-like growth factor I receptor (IGF-1) receptor, and transforming growth factor (TGF)-β₁ receptor. In addition to its catalytic activity, prolidase regulates numerous biological processes. At the cellular level, PEPD acts as a regulator of epidermal growth factor receptor (EGFR) and epidermal growth factor receptor 2 (HER2)-dependent signaling pathways, p53 activity, and expression of the interferon α/β receptor. It is also vital for wound healing: prolidase deficiency having been associated with poor wound healing. Given the desirability of prolidase activation, a study was conducted to assess the influence, if any, of the compounds of the present teaching on prolidase activity.

A series of standards was prepared and 50 μl aliquots of the standards and the test samples identified in Table 6 were added to wells of a prolidase ELISA plate, followed by the addition of 100 μl HDR conjugated anti-prolidase antibody. The plate was then incubated at 37° C. for 1 hour, Following incubation, the ELISA plate was washed and 100 μl of substrate solution was added to each well and the well-plate incubated for 10-15 minutes at room temperature to allow the color generation reaction to occur. At the end of the color generation reaction, 50 μl of stop solution (1 N sulfuric acid) was added to each well and the plates read at 460 nm using a plate reader. The results are presented in Table 6.

### Quantification/Statistical Analysis

To quantify the amount of each substance present, a standard curve was generated using known concentrations of each substance. A regression analysis was performed to establish the line that best fit those data points. Absorbance values for the test materials and untreated samples were used to estimate the amount of each substance present in each sample. Treatment means were compared using ANOVA, with an n+3 per treatment. Statistical significance was set at p≤0.05.

TABLE 6
Prolidase Assay Results

| Treatment | Prolidase in ng/ml |
|---|---|
| Untreated | 74 ± 3 |
| 0.1% DMSO | 72 ± 8 |
| 60 μg/ml Zn di-(di-n-butyryl lysinate) | 145 ± 5* |
| 30 μg/ml Zn di-(di-n-butyryl lysinate) | 115 ± 8* |
| 20 μg/ml Zn di-(di-n-butyryl lysinate) | 104 ± 5* |
| 10 μg/ml Zn di-(di-n-butyryl lysinate) | 66 ± 4 |
| 60 μg/ml Na butyrate | 33 ± 5* |
| 30 μg/ml Na butyrate | 37 ± 5* |
| 20 μg/ml Na butyrate | 47 ± 6* |
| 10 μg/ml Na butyrate | 54 ± 3* |

*Statistically significant difference between treated vs untreated (p ≤ 0.05)

As shown in Table 6, zinc di-(di-n-butyryl lysinate) demonstrated a statistically significant (p<0.05) dose-dependent boosting of prolidase; 50% increase vs control at 60 μg/ml, Surprisingly, in contrast, Na butyrate demonstrated a statistically significant (p<0.05) dose-dependent reduction in prolidase: 50% decrease vs control at 60 μg/ml.

### Example 22: Modulating Endocannabinoid System

Several enzyme activity assessments were performed focused on enzymes associated with the Endocannabinoid System (ECS) and the effect, if any, of the compounds of the present teaching in inhibiting and/or activating enzyme activity, and hence, impacting the ECS itself, Specifically, expression of FAAH, FABP-3, FABP-5 and MAGL enzyme inhibitory activity using the test materials, including compounds of the present teaching, were evaluated. Each study was performed in duplicate and the results averaged.

FAAH—A 0.1 gram sample of each test material was dissolved in 1 mL DMSO to make a stock solution, each of which was subject to 1 to 5 serial dilutions using DMSO for use in determining the IC₅₀. A fluorescence-based assay was used to detect FAAH activity using AMC Arachidonoyl Amide as the substrate. The FAAH enzyme hydrolyzes the substrate, resulting in the liberation of the highly fluorescent 7-amino 4-methyl coumarin (AMC), which is monitored at an excitation wavelength of 355 nm and emission wavelength at 460 nm. The results are presented in Table 7.

FABP-3 and FABP-5—A 0.1 gram sample of each test material was dissolved in 1 mL DMO to make a stock solution, each of which was subject to 1 to 2 serial dilutions using buffer for use in determining the IC₅₀. 50 μL of each test sample and 50 μL of 200 ng/mL of FABP-5 (Raybiotech #268-10276-1) or FABP-3 (Neovateinbio #PT-452) was added per well for analysis.

Analysis was performed using FABP-5 ELISA Kit (Raybiotech #ELH-FABP-5) or FABP-3 EUSA Kit (Raybiotech #ELH-FABP-3) and following the protocol provided by the supplier. The results are presented in Table 7.

MAGL—A 0.1 gram sample of each test material was dissolved in 1 mL DMSO to make a stock solution, each of which was subject to 1 to 2 serial dilutions using buffer for use in determining the IC₅₀. The final concentration in each well was 10 μl of sample, 5.6 μM MAGL (Cayman Catalog #10007812) and 23.6 μM 2-Arachidonoyl Glycerol (Cayman catalog #62160). The results are presented in Table 7.

Surprisingly, the compounds of the present teaching were found to have multi-targeted modulation of the endocannabinoid system, stimulating barrier function related genes and proteins and reducing proinflammatory transcription factors, enzymes and cytokines. As noted from the results, the compounds of the present teaching maintain AEA and 2-AG levels by inhibiting enzymatic activities of transport enzymes (such as, FABP-3 and FABP-5) and degradation enzymes FAAH and MAGL, thus stimulating the healing effects of Anandamide and 2-AG. These results point towards a strong barrier function building, which is the single most important factor in maintaining skin health and longevity.

TABLE 7
FAAH, FABP-3, FABP-5 and MAGL, Inhibitory Activity*

| Present | FAAH | FABP-3 | FABP-5 | MAGL |
|---|---|---|---|---|
| Inventive | IC₅₀ in | IC₅₀ in | IC₅₀ in | IC₅₀ in |
| Compounds | μg/ml | μg/ml | μg/ml | μg/ml |
| Zn di-(di-n- | 368.7 | 242.4 | 644 | 88.8 |
| butyryl lysinate) | | | | |
| Ca di-(di-n- | 213.6 | 188.6 | 786 | 37.6 |
| butyryl lysinate) | | | | |
| Zn dilysinate | 791.8 | 255.2 | 3,014 | 207.0 |
| Ca dilysinate | 445.1 | 208.6 | 871.4 | 856.0 |
| Mg di-(di-n- | 368.5 | 282.5 | 349.1 | 101 |
| butyryl lysinate) | | | | |
| Zn di-(butyryl | 41.3 | 630.9 | 390.5 | 88.3 |
| leucinate) | | | | |
| Sodium butyrate | 805.0 | Not done | Not done | 605.0 |
| CBD¹ | 27.7 | 228.5 | 246.8 | 4,130² |

¹Taken from JID Innovations, 2023, 3: 100178
²Sytheon data

### Example 23: Skin Barrier Repair and Hydration

Several gene expression assays were performed focused on genes associated with skin barrier function and hydration.

The experiments were performed using EpiDermFT (Mattek full thickness) dermal substitutes. Sequencing data was generated by Azenta Life Sciences (HiSeq 2×150 bp) and analyses performed using raw FastQ files provided by Azenta. Differential expression testing was performed for comparison of the test materials, zinc dk-(di-n-butyryl lysinate) (ZDBL) and calcium di-(di-n-butyryl lysinate) (CDBL) versus control.

Although additional work is underway for further validation, the study results demonstrate that ZDBL strongly increased the activity of late cornified envelope 3D (LCE3D), late cornified envelope 3E (LCE3E), small protein reach repeat (SPRR28) and S100 calcium binding protein P (S100P) and CDBL strongly increased the activity of late cornified envelop (LCE2D) and late cornified envelope (LCE3A). Both ZDBL and CDBL increased expression of almost all late differentiation marker genes, such as FLG, IVL, LOR, SPINK5, and TGM1 with ZDBL also increasing the expression of several early differentiation marker genes including DSC1, KRT1, and KRT10. On the other hand, the expression of FABP3 was repressed by both ZDBL and CDBL.

### Example 24: Inflammation—NF-κB and COX-2

Acute and chronic inflammation can have damaging effects on cells, tissues and organs. While the acute inflammation response is typically good and desirable, an excessive inflammatory response can lead to adverse effects. Similarly, where there are instances where the acute inflammatory response is not needed, or at least not to the extent manifested, e.g., sunburns, Hence mitigation of acute inflammation is oftentimes a desirable outcome. On the other hand, chronic inflammation is generally undesirable and leads to several disease conditions involving multiple organs, including the skin and skin function. For the maintenance of immune homeostasis and the prevention of chronic inflammation in epithelial tissues, finely balanced NF-κB activity in both epithelial and immune cells is critical. Similarly, for controlling inflammation in general, inhibiting pro-inflammatory biomarkers, including COX-2, PGE-2 and IL-6, is seen as critical. Further, with COX-2 being associated with increased tumor incidence and promotion of tumor growth and metastasis, COX-2 inhibition has several other benefits, particularly in relation to cancer and the like. In following, a several inhibitory assays were conducted to assess the impact, if any, of the compounds of the present teaching on NF-κB and COX-2 activity.

### NF-κB Inhibitory Effects

An assay was performed in accordance with the protocol of Yang et. al., (In-vitro total antioxidant capacity and anti-inflammatory activity of three common oat-derived avenanthramides, Food Chemistry, 160:338-345, 2014), using Murine myoblast C2C12 cells transfected with an NFκB & luminance reporter gene and 1% DMSO as the control and TNFα as the activator. NFκB expression was quantified from the luminance signals released from cells treated with the test products and the control (untreated).

Zinc di-(di-n-butyryl lysinate) manifested an IC₅₀ of 70.4 μg/ml which was about 110-fold more effective than Na butyrate (IC₅₀ of 7,695 μg/ml) in inhibiting NFκB: demonstrating a strong inhibitory effect.

### COX-2 Inhibitory Effects

A 0.1 gram sample of each test material was dissolved in 1 mL propanediol make a stock solution, each of which was subject to 1 to 2 serial dilutions using buffer for use in determining the IC₅₀ Testing and analysis was performed in accordance with the protocol of the COX-2 Kt (Cayman Catalog #701080) (Cayman catalog #62160). Cannabidiol (CBD) and Na butyrate were used as the control in view their know inhibition of inflammation, CBD is recognized as a strong inhibitor of COX-1 and COX-2 enzyme and widely used for treatment for chronic inflammation. Na butyrate is also known, but it does not have nearly the same adoption as CBD, primarily due to the above-mentioned odor issue. The results are presented in Table 8.

TABLE 8
COX-2 Inhibition

| Test material | COX-2 (IC₅₀ in μg/ml) |
|---|---|
| Zn di-(di-n-butyryl lysinate) | 82.3 |
| Ca di-(di-n-butyryl lysinate) | 111.0 |
| Na butyrate | 294.0 |
| Cannabidiol (CBD) | 1,192 |

As indicated in Table 8, both compounds according to the present teaching, namely Zn di-(di-n-butyryl lysinate) and Ca di-(di-n-butyryl lysinate) had a surprising and marked inhibitory effect on COX-2 enzyme inhibition.

### Formulations

The following present exemplary topical product formulations using the preferred compounds of the present teaching. In each formulation the “New Active” is selected from zinc di-(di-n-butyryl lysinate), zinc di-(di-isobutyryl lysinate), zinc di-(n-butyryl lysinate), zinc di-(isobutyryl lysinate), calcium di-(di-n-butyryl lysinate), calcium di-(di-isobutyryl lysinate), calcium di-(n-butyryl lysinate), calcium di-(isobutyryl lysinate), zinc dilysinate, calcium dilysinate, manganese di-(di-n-butyryl lysinate), manganese di-(di-isobutyryl lysinate), manganese di-(n-butyryl lysinate), manganese di-(isobutyryl lysinate), magnesium di-(di-n-butyryl lysinate), magnesium di-(di-isobutyryl lysinate), magnesium di-(n-butyryl lysinate), magnesium di-(isobutyryl lysinate), zinc di-(n-butyryl leucinate), and zinc di-(isobutyryl leucinate) and mixtures of any two or more of the foregoing; most preferably zinc di-(di-n-butyryl lysinate), zinc di-(di-isobutyryl lysinate), calcium di-(di-n-butyryl lysinate), and calcium di-(di-isobutyryl lysinate) and mixtures of any two or more thereof.

### Formulation 1: Skin Lotion

Formulation 1

| INCI name | Trade Name/Supplier | % w/w |
|---|---|---|
| Phase A | | |
| Water | | 71.50 |
| Glycerin | Glycerin/Acme-Hardesty | 2.00 |
| Phenoxyethanol | Jeechem Phenoxy/Jeen | 1.00 |
| Water & Citric Acid | 10% Citric Acid | 0.75 |
| | Sln/Jungbunzlauer | |
| Trisodium Ethylenediamine | Natrlquest E30/Innospec | 0.10 |
| Disuccinate | | |
| Phase B | | |
| Xanthan Gum | Vanzan NF/Vanderbilt | 0.40 |
| Hydroxyethyl | Sepinov EMT 10/Seppic | 1.00 |
| Acrylate/Sodium | | |
| Acryloyldimethyl | | |
| Taurate Copolymer | | |
| Phase C | | |
| Helianthus Annuus | Sunflower Oil/Jeen | 5.00 |
| (Sunflower) Seed Oil | | |
| Tocopheryl Acetate | Vitamin E Acetate/Jeen | 0.25 |
| Cetearyl Alcohol & | Ritapro 300/Rita | 2.50 |
| Ceteareth-20 | | |
| Potassium Cetyl Phosphate | Amphisol K/DSM | 1.50 |
| Butyrospermum Parkii | Shebu Refined/Rita | 2.00 |
| (Shea) Butter | | |
| Isosorbide Dicaprylate | Hydra Synol ® DOI/Sytheon | 2.00 |
| Phase D | | |
| Butylene Glycol | Jeechem BUGL/Jeen | 8.00 |
| New Active | Sytheon | 2.00 |
| Total | | 100.00 |

This formulation is prepared by first adding the components of Phase A to a main kettle equipped with a homogenizer. Thereafter, the Phase B components are slowly sprinkled into the Phase A material. Once the former is fully dispersed in the latter, the mixture is heated to 65-70° C. Concurrently, the components of Phase Care placed and mixed in a side kettle and heated to 70-75° C. Once both mixtures are to the proper temperatures, Phase C is added to the Phase AR mix and the mixture mixed for 15-20 minutes. Concurrently, the components of Phase D are combined in a side kettle and heated to 75° C. Once the New Active is dissolved, Phase D is added to the combined Phase ABC and mixed for 5-10 minutes. Once the mixture is homogenous, mixing is switched to side sweep mixing and the mixture allowed to cool to room temperature, Notes: pH—4.5-5.0; Viscosity: Spindle-TE, S95; Speed—0.3 rpm; Range—200,000-500,000 mPas

### Formulation 2: Moisturizing Serum

Formulation 2

| INCI name | Trade Name/Supplier | % w/w |
|---|---|---|
| Phase A | | |
| Water | | 76.65 |
| Glycerin | Glycerin/Acme-Hardesty | 3.00 |
| Water & Potassium | Euxyl K 712/Schulke | 1.00 |
| Sorbate & Sodium Benzoate | | |
| Water & Citric Acid | 10% Citric Acid | 1.00 |
| | Sln/Jungbunzlauer | |
| Trisodium Ethylenediamine | Natrlquest E30/Innospec | 0.10 |
| Disuccinate | | |
| Phase B | | |
| Acacia Senegal Gum & | Solagum AX/Seppic | 0.50 |
| Xanthan Gum | | |
| Hydroxyethyl | Sepinov EMT 10/Seppic | 1.50 |
| Acrylate/Sodium | | |
| Acryloyldimethyl | | |
| Taurate Copolymer | | |
| Phase C | | |
| Helianthus Annuus | Sunflower Oil/Jeen | 3.00 |
| (Sunflower) Seed Oil | | |
| Isostearyl Alcohol & | Emulfree CBG/Gattefosse | 1.00 |
| Butylene Glycol Cocoate & | | |
| Ethylcellulose | | |
| Isosorbide Dicaprylate | Hydra Synol ® DOI/Sytheon | 3.00 |
| Phase D | | |
| Propanediol | Zemea/DuPont | 8.00 |
| New Active | Sytheon | 2.00 |
| Total | | 100.00 |

Formulation 2 is prepared by combining the Phase A ingredients in a kettle equipped with a homogenizer and mixed. As Phase A is being mixed, the components of Phase B are sprinkled in and the combination mixed until uniform, Concurrently, the components of Phase Care mixed in a side kettle until uniform and then added to the Phase AB mix and this mixture mixed for 10-15 minutes. The Phase D ingredients are combined in a in a side kettle and heated to 75° C. to dissolve the New Active. Once dissolved, heating is stopped and the mixture allowed to cool to room temperature following which it is added to the Phase ABC mix and the mixture mixed for 5-10 minutes. Notes: pH—5.5-6.0; Viscosity: Spindle-TD, 94, Speed—2.0 rpm; Range—8,000-11,000 mPas.

### Formulation 3: Skin Even Toning Lotion

Formulation 3

| INCI name | Trade Name/Supplier | % w/w |
|---|---|---|
| Phase A | | |
| Water | | 70.00 |
| Glycerin | Glycerin/Acme-Hardesty | 2.00 |
| Phenoxyethanol | Jeechem Phenoxy/Jeen | 1.00 |
| Water & Citric Acid | 10% Citric Acid | 0.75 |
| | Sln/Jungbunzlauer | |
| Trisodium Ethylenediamine | Natrlquest E30/Innospec | 0.10 |
| Disuccinate | | |
| Phase B | | |
| Xanthan Gum | Vanzan NF/Vanderbilt | 0.40 |
| Hydroxyethyl | Sepinov EMT 10/Seppic | 1.00 |
| Acrylate/Sodium | | |
| Acryloyldimethyl | | |
| Taurate Copolymer | | |
| Phase C | | |
| Helianthus Annuus | Sunflower Oil/Jeen | 5.00 |
| (Sunflower) Seed Oil | | |
| Tocopheryl Acetate | Vitamin E Acetate/Jeen | 0.25 |
| Cetearyl Alcohol & | Ritapro 300/Rita | 2.50 |
| Ceteareth-20 | | |
| Potassium Cetyl Phosphate | Amphisol K/DSM | 1.50 |
| Butyrospermum Parkii | Shebu Refined/Rita | 2.00 |
| (Shea) Butter | | |
| Isosorbide Dicaprylate | Hydra Synol ® DOI/Sytheon | 2.00 |
| Phase D | | |
| Butylene Glycol | Jeechem BUGL/Jeen | 8.00 |
| New Active | Sytheon | 2.00 |
| Hexylresorcinol | Synovea ® HR/Sytheon | 0.50 |
| Total | | 100.00 |

Formulation 3 is prepared by first adding the components of Phase A to a main kettle equipped with a homogenizer. Thereafter, the Phase B components are slowly sprinkled into the Phase A material. Once the former is fully dispersed in the latter, the mixture is heated to 65-70° C. Concurrently, the components of Phase Care placed and mixed in a side kettle and heated to 70-75° C. Once both mixtures are to the proper temperatures, Phase C is added to the Phase AB mix and the mixture, mixed for 15-20 minutes. Concurrently, combine the first two components of Phase D in a side kettle and heat to 75° C. Once the New Active is dissolved the mixture is cooled to 45° C. and the hexylresorcinol is added and mixed until uniform. The combined Phase ABC mix is then switched to side sweep mixing and the mixture allowed to cool 45° C. Thereafter, Phase D is added to the Phase ABC mix and mixed for 5-10 minutes and the total mixture allowed to cool to room temperature. Notes: pH—4.5-5.0; Viscosity: Spindle-TE, S95; Speed—0.3 rpm; Range—200,000-500,000 mPas.

### Formulation 4: Skin Brightening Lotion

Formulation 4

| INCI name | Trade Name/Supplier | % w/w |
|---|---|---|
| Phase A | | |
| Water | | 70.50 |
| Glycerin | Glycerin/Acme-Hardesty | 2.00 |
| Phenoxyethanol | Jeechem Phenoxy/Jeen | 1.00 |
| Water & Citric Acid | 10% Citric Acid | 0.75 |
| | Sln/Jungbunzlauer | |
| Trisodium Ethylenediamine | Natrlquest E30/Innospec | 0.10 |
| Disuccinate | | |
| Phase B | | |
| Xanthan Gum | Vanzan NF/Vanderbilt | 0.40 |
| Hydroxyethyl | Sepinov EMT 10/Seppic | 1.00 |
| Acrylate/Sodium | | |
| Acryloyldimethyl | | |
| Taurate Copolymer | | |
| Phase C | | |
| Helianthus Annuus | Sunflower Oil/Jeen | 5.00 |
| (Sunflower) Seed Oil | | |
| Tocopheryl Acetate | Vitamin E Acetate/Jeen | 0.25 |
| Cetearyl Alcohol & | Ritapro 300/Rita | 2.50 |
| Ceteareth-20 | | |
| Potassium Cetyl Phosphate | Amphisol K/DSM | 1.50 |
| Butyrospermum Parkii | Shebu Refined/Rita | 2.00 |
| (Shea) Butter | | |
| Isosorbide Dicaprylate | Hydra Synol ® DOI/Sytheon | 2.00 |
| Bakuchiol | Sytenol ® A/Sytheon | 1.00 |
| Phase D | | |
| Butylene Glycol | Jeechem BUGL/Jeen | 8.00 |
| New Active | Sytheon | 2.00 |
| Total | | 100.00 |

Formulation 4 is prepared by first adding the components of Phase A to a main kettle equipped with: a homogenizer. Thereafter, the Phase B components are slowly sprinkled into the Phase A material. Once the former is fully dispersed in the latter, the mixture is heated to 65-70° C. Concurrently, the components of Phase Care placed and mixed in a side kettle and heated to 70-75° C. Once both mixtures are to the proper temperatures, Phase Cis added to the Phase AB mix and the mixture mixed for 15-20 minutes. Concurrently, the components of Phase D are combined in a side kettle and heated to 75° C., Once the New Active is dissolved, Phase D is added to the combined Phase ABC and mixed for 5-10 minutes. Once the mixture is homogenous, mixing is switched to side sweep mixing and the mixture allowed to cool to room temperature. Notes: pH—4.5-5.0 Viscosity: Spindle-TE, S95; Speed—0.3 rpm; Range—200,000-500,000 mPas.

### Formulation 5: Skin Anti-Aging Lotion

Formulation 5

| INCI name | Trade Name/Supplier | % w/w |
|---|---|---|
| Phase A | | |
| Water | | 65.50 |
| Glycerin | Glycerin/Acme-Hardesty | 2.00 |
| Phenoxyethanol | Jeechem Phenoxy/Jeen | 1.00 |
| Water & Citric Acid | 10% Citric Acid | 0.75 |
| | Sln/Jungbunzlauer | |
| Trisodium Ethylenediamine | Natrlquest E30/Innospec | 0.10 |
| Disuccinate | | |
| Phase B | | |
| Xanthan Gum | Vanzan NF/Vanderbilt | 0.40 |
| Hydroxyethyl | Sepinov EMT 10/Seppic | 1.00 |
| Acrylate/Sodium | | |
| Acryloyldimethyl | | |
| Taurate Copolymer | | |
| Phase C | | |
| Helianthus Annuus | Sunflower Oil/Jeen | 5.00 |
| (Sunflower) Seed Oil | | |
| Tocopheryl Acetate | Vitamin E Acetate/Jeen | 0.25 |
| Cetearyl Alcohol & | Ritapro 300/Rita | 2.50 |
| Ceteareth-20 | | |
| Potassium Cetyl Phosphate | Amphisol K/DSM | 1.50 |
| Butyrospermum Parkii | Shebu Refined/Rita | 2.00 |
| (Shea) Butter | | |
| Isosorbide Dicaprylate | Hydra Synol ® DOI/Sytheon | 2.00 |
| Acetyl Zingerone | Synoxyl ® AZ/Sytheon | 1.00 |
| Phenethyl Benzoate | X-Tend 226/Ashland | 5.00 |
| Phase D | | |
| Butylene glycol | Jeechem BUGL/Jeen | 8.00 |
| New Active | Sytheon | 2.00 |
| Total | | 100.00 |

### Formulation 5 is Prepared According to the Same Method as Formulation 4.

### Formulation 6: Protective Skin Lotion

Formulation 6

| INCI name | Trade Name/Supplier | % w/w |
|---|---|---|
| Phase A | | RC-04- |
| | | 92.01 |
| Water | | 68.00 |
| Glycerin | Glycerin/Acme-Hardesty | 2.00 |
| Phenoxyethanol | Jeechem Phenoxy/Jeen | 1.00 |
| Water & Citric Acid | 10% Citric Acid | 0.75 |
| | Sln/Jungbunzlauer | |
| Trisodium Ethylenediamine | Natrlquest E30/Innospec | 0.10 |
| Disuccinate | | |
| Phase B | | |
| Xanthan Gum | Vanzan NF/Vanderbilt | 0.40 |
| Hydroxyethyl | Sepinov EMT 10/Seppic | 1.00 |
| Acrylate/Sodium | | |
| Acryloyldimethyl | | |
| Taurate Copolymer | | |
| Phase C | | |
| Helianthus Annuus | Sunflower Oil/Jeen | 5.00 |
| (Sunflower) Seed Oil | | |
| Tocopheryl Acetate | Vitamin E Acetate/Jeen | 0.25 |
| Cetearyl Alcohol & | Ritapro 300/Rita | 2.50 |
| Ceteareth-20 | | |
| Potassium Cetyl Phosphate | Amphisol K/DSM | 1.50 |
| Butyrospermum Parkii | Shebu Refined/Rita | 2.00 |
| (Shea) Butter | | |
| Isosorbide Dicaprylate | Hydra Synol ® DOI/Sytheon | 2.00 |
| Phase D | | |
| Butylene Glycol | Jeechem BUGL/Jeen | 8.00 |
| New Active | Sytheon | 2.00 |
| Phase E | | |
| Terminalia Chebula | Synastol ® TC/Sytheon | 0.50 |
| Fruit Extract | | |
| Water | | 3.00 |
| Total | | 100.00 |

Formulation 6 is prepared by first adding the components of Phase A to a main kettle equipped with a homogenizer. Thereafter, the Phase B components are slowly sprinkled into the Phase A material. Once the former is fully dispersed in the latter, the mixture is heated to 65-70° C., Concurrently, the components of Phase C are placed and mixed in a side kettle and heated to 70-75° C. Once both mixtures are to the proper temperatures, Phase C is added to the Phase AB mix and the mixture mixed for 15-20 minutes, Concurrently, the components of Phase D New Active component is dissolved, Phase D is added to the combined Phase ABC and mixed for 5-10 minutes. The combined Phase ABC mix is then switched to side sweep mixing and the mixture allowed to cool 45° C. Concurrently, the water component of Phase E is added to separate kettle and heated to 45° C. whereupon the Terminalia Chebula extract is added and mixed until dissolved. Thereafter, Phase E is added to the Phase ABCD mix and mixed until uniform and then the batch allowed to cool to room temperature. Notes: pH—4.5-5.0; Viscosity: Spindle-TE, S95; Speed—0.3 rpm; Range—200,000-500,000 mPa.

### Formulation 7 Anti-Aging Cream

Formulation 7

| INCI name | Trade Name/Supplier | % w/w |
|---|---|---|
| Phase A | | |
| Water | | 71.65 |
| Glycerin | Glycerin/Acme-Hardesty | 2.00 |
| Disodium EDTA | Ronacare EDTA/EMD | 0.10 |
| Water & Citric Acid | 10% Citric Acid | 0.50 |
| | Sln/Jungbunzlauer | |
| Steareth-2 | Jeecol SA-2/Jeen | 1.25 |
| PEG-8 | Pluracare E 400/BASF | 2.00 |
| Niacinamide | Niacinamide/Lonza | 2.00 |
| Caffeine | Caffeine/EMD | 0.50 |
| Butylene Glycol | Jeechem BUGL/Jeen | 3.00 |
| Caprylyl Glycol & | Jeecide CAP-2/Jeen | 1.00 |
| Phenoxyethanol & | | |
| Hexylene Glycol | | |
| Phase B | | |
| Ammonium | Aristoflex | 1.00 |
| Acryloyldimethyltaurate/ | AVC/Clariant | |
| VP Copolymer | | |
| Phase C | | |
| Cyclopentasiloxane | Xiameter PMX-0245/ | 5.00 |
| | Dow Corning | |
| Dimethicone | Dimethicone Fluid/ | 1.00 |
| | Making Cosmetics | |
| Isosorbide Dicaprylate | Hydra Synol ® | 2.00 |
| | DOI/Sytheon | |
| Phase D | | |
| Denatured Alcohol | Denatured Alcohol/ | 4.00 |
| | Quality Chemical | |
| New Active | Sytheon | 1.00 |
| Phase E | | |
| Hydroxyethyl Acrylate/ | Simulgel NS/Seppic | 2.00 |
| Sodium Acryloyldimethyl | | |
| Taurate Copolymer & | | |
| Squalane & Polysorbate 60 | | |
| Total | | 100.00 |

Formulation 7 is prepared by combining the Phase A ingredients in a kettle equipped with a homogenizer and mixed. As Phase A is being mixed, the components of Phase B are sprinkled in and the combination mixed until uniform. Concurrently, the components of Phase C are mixed in a side kettle until uniform and then added to the Phase AB mix and this mixture mixed for 10-15 minutes. The Phase D ingredients are combined in a side kettle and mixed until uniform. Once the New Active is dissolved (minor heating may be necessary to facilitate dissolving) Phase D is added to the Phase ABC mix and the mixture mixed for 5-10 minutes. Thereafter, Phase E is added and mixed until uniform. Notes: pH—5.5-6.0; Viscosity: Spindle-TE, S95; Speed—0.3 rpm; Range—400,000-700,000 mPas.

### Formulation 8: Skin Lightening Lotion

Formulation 8 is prepared by combining the Phase A ingredients in a kettle equipped with a homogenizer and mixed. As Phase A is being mixed, the components of Phase B are sprinkled in and the combination mixed. Once Phase B is dispersed, the mixture is heated to 65-70° C., Concurrently, the components of Phase C are mixed in a side kettle and heated to 70-75° C. until uniform. Once both Phase AB and Phase C are at the indicated temperatures, Phase C is added to Phase AB and mixed for 15-20 minutes. The Phase D ingredients are combined in a side kettle and mixed until uniform (minor heating may be necessary to facilitate dissolving of the New Active. The Phase ABC mix is changed to a side sweep mixing and cooled to 45° C. and Phase U added with mixing. Phase E is prepared in a side kettle and mixed until uniform, Phase E is then added to the Phase ABCD and mixed for 5-10 minute after which it is cooled to room temperature. Notes: pH—4.5-5.0; Viscosity: Spindle-TE, S95; Speed—0.3 rpm; Range—200,000-500,000 mPas.

Formulation 8

| INCI name | Trade Name/Supplier | % w/w |
|---|---|---|
| Phase A | | |
| Water | | 72.00 |
| Glycerin | Glycerin/Acme-Hardesty | 2.00 |
| Phenoxyethanol | Jeechem Phenoxy/Jeen | 1.00 |
| Water & Citric Acid | 10% Citric Acid | 0.75 |
| | Sln/Jungbunzlauer | |
| Trisodium Ethylenediamine | Natrlquest E30/Innospec | 0.10 |
| Disuccinate | | |
| Phase B | | |
| Xanthan Gum | Vanzan NF/Vanderbilt | 0.40 |
| Hydroxyethyl | Sepinov EMT 10/Seppic | 1.00 |
| Acrylate/Sodium | | |
| Acryloyldimethyl | | |
| Taurate Copolymer | | |
| Phase C | | |
| Helianthus Annuus | Sunflower Oil/Jeen | 5.00 |
| (Sunflower) Seed Oil | | |
| Tocopheryl Acetate | Vitamin E Acetate/Jeen | 0.25 |
| Cetearyl Alcohol & | Ritapro 300/Rita | 2.50 |
| Ceteareth-20 | | |
| Potassium Cetyl Phosphate | Amphisol K/DSM | 1.50 |
| Butyrospermum Parkii | Shebu Refined/Rita | 2.00 |
| (Shea) Butter | | |
| Isosorbide Dicaprylate | Hydra Synol ® DOI/Sytheon | 2.00 |
| Phase D | | |
| Coco-Betaine | Cola Teric CB/Colonial | 2.00 |
| | Chemical | |
| Cocamidopropyl Betaine | Dehyton PK 45/BASF | 2.00 |
| New Active | Sytheon | 1.00 |
| Phase E | | |
| Butylene Glycol | Jeechem BUGL/Jeen | 4.00 |
| Hexylresorcinol | Synovea ® HR/Sytheon | 0.50 |
| Total | | 100.00 |

### Formulation 9: Anti-Aging Lotion

Formulation 9 is prepared by combining the Phase A ingredients in a kettle equipped with a homogenizer and mixed. As Phase Ais being mixed, the components of Phase B are sprinkled in and the combination mixed. Once Phase B is dispersed, the mixture is heated to 65-70° C. Concurrently, the components of Phase C are mixed in a side kettle and heated to 70-75° C. until uniform. Once both Phase AS and Phase C are at the indicated temperatures, Phase C is added to Phase AB and mixed for 15-20 minutes. The Phase D ingredients are combined in a side kettle and mixed until uniform (minor heating may be necessary to facilitate dissolving of the New Active. The Phase ABC mix is changed to a side sweep mixing and cooled to room temperature, Once at room temperature Phase D is added and mixed for 5-10 minutes. Notes: pH—4.5-5.0; Viscosity: Spindle-TE, S95; Speed—0.3 rpm; Range—200,000-500,000 mPas.

Formulation 9

| INCI name | Trade Name/Supplier | % w/w |
|---|---|---|
| Phase A | | RC-04- |
| | | 95.02 |
| Water | | 70.50 |
| Glycerin | Glycerin/Acme-Hardesty | 2.00 |
| Phenoxyethanol | Jeechem Phenoxy/Jeen | 1.00 |
| Water & Citric Acid | 10% Citric Acid | 0.75 |
| | Sln/Jungbunzlauer | |
| Trisodium Ethylenediamine | Natrlquest E30/Innospec | 0.10 |
| Disuccinate | | |
| Phase B | | |
| Xanthan Gum | Vanzan NF/Vanderbilt | 0.40 |
| Hydroxyethyl | Sepinov EMT 10/Seppic | 1.00 |
| Acrylate/Sodium | | |
| Acryloyldimethyl | | |
| Taurate Copolymer | | |
| Phase C | | |
| Helianthus Annuus | Sunflower Oil/Jeen | 5.00 |
| (Sunflower) Seed Oil | | |
| Tocopheryl Acetate | Vitamin E Acetate/Jeen | 0.25 |
| Cetearyl Alcohol & | Ritapro 300/Rita | 2.50 |
| Ceteareth-20 | | |
| Potassium Cetyl Phosphate | Amphisol K/DSM | 1.50 |
| Butyrospermum Parkii | Shebu Refined/Rita | 2.00 |
| (Shea) Butter | | |
| Isosorbide Dicaprylate | Hydra Synol ® DOI/Sytheon | 2.00 |
| Acetyl Zingerone | Synoxyl ® AZ/Sytheon | 1.00 |
| Phenethyl Benzoate | X-Tend 226/Ashland | 5.00 |
| Phase D | | |
| Denatured Alcohol | Denatured Alcohol/ | 4.00 |
| | Quality Chemical | |
| New Active | Sytheon | 1.00 |
| Total | | 100.00 |

Without further elaboration, it is believed that one skilled in the art, using the preceding description, can utilize the present invention to its fullest extent. Furthermore, while the present invention has been described with respect to the aforementioned specific embodiments and examples, it should be appreciated that other embodiments, changes and modifications utilizing the concept of the present invention are possible, and within the skill of one in the art, without departing from the spirit and scope of the invention. The preceding preferred specific embodiments are, therefore, to be construed as merely illustrative, and not limitative of the remainder of the disclosure in any way whatsoever.

I claim:

1. A method of mitigating or reversing the effects of aging and/or environmental impacts upon the skin and/or its physiological processes; reversing the adverse effects of skin maladies and diseases; rejuvenating, enhancing and/or improving the look, feel, integrity and/or physiological processes of the skin, said method comprising topically applying to the skin a composition comprising an effective amount for achieving the foregoing objectives of at least one compound according to Structure I: 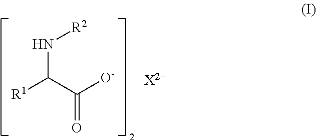
wherein:
X is Zn, Mg or Mn;
each R¹ is independently selected from the group consisting of —(CH₂)₄NHR³, —CH₂CH(CH₃)₂, —CH(CH₃)CH₂CH₃, and —CH(CH₃)₂;
each R² is independently selected from the group consisting of —H, —COCH₃, —COCH₂CH₃, —COCH₂CH₂CH₃, —COCH(CH₃)₂; —COCH₂CH₂COOCH₃; —COCH(COOCH₃)₂ and —COCH(CH₄)COOCH₃;
each R³ is independently selected from the group consisting of —H, —COCH₃, —COCH₂CH₃, —COCH₂CH₂CH₃, —COCH(CH₃)₂; —COCH₂CH₂COOCH₃; —COCH(COOCH₃)₂ and —COCH(CH₃)COOCH₃;
provided that at least one of R² and R³ is not H

2. The method of claim 1 wherein X is Zn.

3. The method of claim 1 wherein X is Mg.

4. The method of claim 1 wherein each R¹ is the same and each R² is the same.

5. The method of claim 1 wherein each R² is the same and each R³ is the same.

6. The method of claim 1 wherein each R² is —H.

7. The method of claim 1 wherein each R¹ is —(CH₂)₄NHR³.

8. The method of claim 1 wherein each R¹ is —(CH₂)₄NHR³ and each R³ is the same and is selected from —COCH₃, —COCH₂CH₃, —COCH₂CH₂CH₃, and —COCH(CH₃)₂.

9. The method of claim 8 wherein all R²s and R³s are the same.

10. The method of claim 8 wherein the R²s are H.

11. The method of claim 10 where the R³s are —COCH₂CH₂CH₃.

12. The method of claim 8 wherein the R³s are —COCH₂CH₂CH₃ or —COCH(CH₃)₂.

13. The method of claim 8 wherein the R²s and R³s are —COCH₂CH₂CH₃.

14. The method of claim 1 wherein the compound of Structure 1 is (a) zinc di-(di-n-butyryl lysinate), depicted as 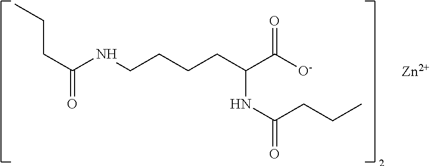
(b) zinc di-(di-isobutyryl lysinate), depicted as 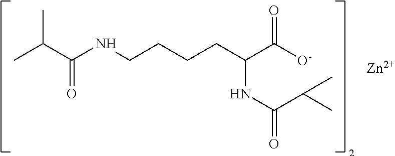
or a mixture of the two.

15. The method of claim 1 wherein the compound of Structure 1 is zinc di-(di-n-butyryl lysinate).

16. The method of claim 1 wherein the composition further comprises a dermatologically acceptable carrier.

17. The method of claim 16 wherein the dermatologically acceptable carrier is a formulated cosmetic, sunscreen, moisturizer, skin serum, anti-aging composition, acne treatment, general skin care product, skin rejuvenation product, topical pharmaceutical product, or sunburn treatment product and the compound according to Structure 1 is present in an amount of from about 0.01 to about 10 percent by weight based on the weight of the composition.

18. The method of claim 16 wherein the composition further comprises at least one of acetyl zingerone, bakuchiol, isosorbide dicaprylate, Terminalia chebula fruit extract, ethyl linoleate, isosorbide dilinoleate, and isosorbide disunflowerseedate in an amount of from 0.01 to about 20 weight percent based on the weight of the composition.

19. The method of claim 1 wherein the composition is applied to those areas of the skin manifesting skin damage due to disease, maladies, and/or environmental factors.

20. The method of claim 1 wherein the composition is applied to those areas of the skin manifesting skin damage due to chronological aging.
